(19) United States
(12) Patent Application Publication
(10) Pub. No.: US 2026/0217716 A1
(43) Pub. Date: Jul. 30, 2026

(54) ANDROGEN RECEPTOR MODULATORS AND METHODS FOR THEIR USE

(71) Applicant: Capulus Therapeutics, LLC, San Francisco, CA (US)

(72) Inventors: Michael John Green, Half Moon Bay, CA (US); Naveen Naganaboina, Desaipet, Warangal (IN); Ravikumar Muttineni, Hyderabad (IN); Sumana Ghosh, Delhi (IN)

(21) Appl. No.: 19/141,794

(22) PCT Filed: Dec. 21, 2023
(86) PCT No.: PCT/US2023/085401

§ 371 Date: Jun. 20, 2025

Related U.S. Application Data
(60) Provisional application No. 63/476,445, filed on Dec. 21, 2022.

(51) Int. Cl. C07D 487/04; A61K 31/5025

(52) U.S. Cl. CPC C07D 487/04; A61K 31/5025

(57) ABSTRACT

Androgen receptor modulators, and more particularly N-terminal domain androgen receptor inhibitors, comprising a heterocyclic bicyclic [1,2,4]triazolo[4,3-b]pyridazinyl ring core, as well as pharmaceutical compositions thereof are disclosed. Such compounds target the N-terminal domain (NTD) of the androgen receptor (AR) for treating cancers such as prostate cancer and specifically metastatic castration-resistant prostate cancer (mCRPC).

### CROSS REFERENCE TO RELATED APPLICATION

This application claims a benefit of, and priority to, U.S. Provisional Patent Application No. 63/476,445, filed Dec. 21, 2022, the disclosure of which is incorporated by reference in its entirety.

### TECHNICAL FIELD

Embodiments of the present invention generally relate to androgen receptor modulators, and more particularly to N-terminal domain androgen receptor inhibitors.

### BACKGROUND

Androgens mediate their effects through the androgen receptor (AR). Androgens play a role in a wide range of developmental and physiological responses and are involved in male sexual differentiation, maintenance of spermatogenesis, and male gonadotropin regulation. The androgen receptor (AR) is encoded by the NR3C4 gene and is within the nuclear receptor family. The binding of AR to its endogenous ligand 5α-dihydrotestosterone (DHT) and testosterone is responsible for starting male sexual development and differentiation. Testosterone is synthesized by the Leydig cells in the testes and circulates mostly bound to the serum sex hormone-binding globulin (SHBG) and albumin. The free form enters prostate cells, where testosterone is converted to the more potent form of DHT, which promotes growth and survival of prostate cells. High affinity binding of DHT to AR displaces other AR bound proteins and drives the AR complex to the nucleus where it dimerizes and binds to androgen response elements (AREs) in the promoter region of target genes. The full-length AR protein has three major domains, the N-terminal domain (NTD), the DNA-binding domain (DBD) and the ligand-binding domain (LBD). All three are required for proper regulation and function of AR. The AR can be activated in the absence of testicular androgens by alternative signal transduction pathways in castration-resistant disease, which is consistent with the finding that nuclear AR protein is present in secondary prostate cancer tumors.

Prostate cancer is one of the most common male malignancies and is the second leading cause of cancer deaths among males in the United States. The current standard of care is androgen deprivation therapy, which is initially effective for treatment of metastatic prostate cancer, but most patients develop castration-resistant prostate cancer (CRPC) that can further develop into metastatic CRPC (mCRPC).

Current AR targeting agents available for treatment primarily target the LBD of AR. However, point mutations, gene rearrangements resulting in overexpression of AR, development of constitutively active AR structural variants, and several other mechanisms may render AR-LBD targeting inhibitors ineffective. Multiple mechanisms may lead to resistance in prostate cancer cells, one of them being increased production of short AR splice variants. The most prominent variants in prostate and breast cancer are ARV7 and ARV567es.

Constitutively active AR structural variants (without LBD), of which AR splice variant-7 (AR-V7) is the most frequently detected, is considered a potential driver of castration-resistant prostate cancer (CRPC). AR-V7 is expressed in over 80% of CRPC patient samples in Stand Up to Cancer (SU2C) prostate cancer cohort. AR-V7 can dimerize without the presence of the receptor ligand and drive AR response genes transcription in the nucleus, this provides them with the ability to drive prostate cancer even in the absence of LBD. ARV7 displays non canonical nuclear import kinetics and short chromatin residence time as compared to ARFL. Further, in-vitro and in-vivo studies have shown that AR-V7 can drive prostate cancer and its inhibition leads to control tumor growth and induces tumor regression. Further inhibition of AR structural variants in combination with AR-antagonist targeting AR-LBD could enhance therapeutic efficacy.

The AR pathway has also been implicated in breast cancer and may be a suitable target for triple negative breast cancer (TNBC) where AR plays a role in the proliferation of breast cancer cells by either promoting proliferation or inhibiting proliferation depending on the expression of estrogen receptor (ER) and human epidermal growth factor receptor 2 (HER2), as well as ovarian cancers. Up to 90% of breast cancer have AR expression and AR-V7 has been detected in primary breast cancer samples and cell lines.

Therefore, it is desirable to target domains other than LBD as a potential path for treating cancers such as prostate, breast and ovarian cancers.

### SUMMARY

The following summary is illustrative only and is not intended to be in any way limiting. In addition to the illustrative aspects, example embodiments, and features described, further aspects, example embodiments, and features will become apparent by reference to the following detailed description.

In some aspects of the present invention, a compound having a structure of formula (I), or a pharmaceutically acceptable salt thereof is presented. Formula (I) is:

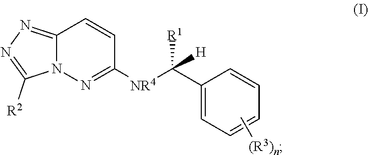

wherein
R¹ and R² are independently a C₁-C₆ alkyl group optionally substituted with one or more fluorine or a C₃-C₆ cycloalkyl group optionally substituted with one or more fluorine;
R³ is independently at each occurrence halogen, —CN, —OR⁵, —C(═O)R⁵, —NHC(═O)R⁵, —C(═O)NR⁵R⁶, —NR⁵R⁶, —P(═O)R⁵R⁶, a C₁-C₆ alkyl group optionally substituted with one or more fluorine, or a C₃-C₆ cycloalkyl group optionally substituted with one or more fluorine;
R⁴ is hydrogen or a C₁-C₆ alkyl group;
R⁵ and R⁶ are independently at each occurrence hydrogen, a C₁-C₆ alkyl group, or R⁵ and
R⁶ together with the nitrogen to which each is attached form a C₃-C₆ heterocycloalkyl group optionally substituted with one or more fluorine; and
“n” is an integer from 1 to 5.

In some aspects of the present invention, a pharmaceutical composition is presented. The pharmaceutical composition includes a compound having a formula (I) or a pharmaceutically acceptable salt, solvate, hydrate, polymorph, or co-crystal thereof, and a pharmaceutically carrier, diluent, or excipient.

In some aspects of the present invention, a method for modulation of androgen receptor activity for treatment of prostate cancer, breast cancer, ovarian cancer, or melanoma is presented. The method includes administering to a patient a therapeutically effective amount of a pharmaceutical composition including a compound of formula (I).

### DETAILED DESCRIPTION OF EXAMPLE EMBODIMENTS

Various example embodiments will now be described more fully with reference to the accompanying drawings in which only some example embodiments are shown. Specific structural and functional details disclosed herein are merely representative for purposes of describing example embodiments. Example embodiments, however, may be embodied in many alternate forms and should not be construed as limited to only the example embodiments set forth herein. On the contrary, example embodiments are to cover all modifications, equivalents, and alternatives thereof.

The terminology used herein is for the purpose of describing particular example embodiments only and is not intended to be limiting. Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, steps, operations, elements, components and/or groups, but do not preclude the presence or addition of one or more other features, steps, operations, elements, components, and/or groups thereof.

Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

To more clearly and concisely describe and point out the subject matter of the claimed invention, the following definitions are provided for specific terms that are used in the following description and the claims appended hereto.

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Unless otherwise stated, structures depicted herein are also meant to include compounds that differ only in the presence of one or more isotopically enriched atoms. For example, compounds having the present structures including the replacement of hydrogen by deuterium or tritium, the replacement of a carbon by a 13C- or 14C-enriched carbon, or the replacement of a fluorine by a 18F-enriched fluorine, are within the scope of this invention. Such compounds are useful, for example, as analytical tools, as probes in biological assays, or as therapeutic agents in accordance with the present invention.

As described in detail later, embodiments of the present invention describe compounds preferably having an R configuration for the asymmetric center. In some embodiments, compounds of the present invention are substantially free of the corresponding enantiomer (i.e., S enantiomer), and may also be referred to as “optically enriched.” “Optically-enriched,” as used herein, means that the compound is made up of a significantly greater proportion of one enantiomer (i.e., R enantiomer). In certain embodiments, the compound is made up of at least about 90% by weight of the preferred R enantiomer. In other embodiments, the compound is made up of at least about 95%, 98%, or 99% by weight of the preferred R enantiomer. Preferred R enantiomers may be isolated from racemic mixtures by any method known to those skilled in the art, including chiral high-pressure liquid chromatography (HPLC) and the formation and crystallization of chiral salts or prepared by asymmetric syntheses.

As used herein, the term “alkyl group” refers to a saturated monovalent group consisting of a linear or branched array of atoms that is not cyclic. Alkyl groups are defined to include at least one carbon atom and are represented by formula CₙH₂ₙ₊₁. The array of atoms included in the alkyl group may be composed exclusively of carbon and hydrogen. By way of example, the term “C₁-C₁₀ alkyl group” contains at least one but no more than 10 carbon atoms. A methyl group (i.e. CH₃—) is an example of a monovalent C₁ alkyl group. A decyl group (i.e., CH₃(CH₂)₉—) is an example of a monovalent C₁₀ alkyl group. Non-limiting examples of alkyl groups include methyl, ethyl, n-propyl, isopropyl, n-butyl, t-butyl, isobutyl, sec-butyl, n-pentyl, n-hexyl, n-heptyl, n-octyl, and the like.

As used herein the term “cycloalkyl group” refers to a group having a valence of one, and consisting of an array of atoms that is cyclic but which is not aromatic. A “cycloalkyl” may include one or more noncyclic components. For example, a cyclohexylmethyl group (C₆H₁₁CH₂—) is a cycloalkyl group that includes a cyclohexyl ring (the array of atoms that is cyclic but which is not aromatic) and a methylene group (the noncyclic component). By way of example, the term “a C₃-C₁₀ cycloalkyl group” includes cycloalkyl groups containing at least three but no more than 10 carbon atoms. The cyclohexylmethyl group (C₆H₁₁CH₂—) represents a C₇ cycloalkyl group.

As used herein, the term “heterocycloalkyl group” refers to non-aromatic group having a valence of one which consists of at least one heteroatom selected from the group consisting of nitrogen, oxygen and sulfur. Unless stated otherwise specifically in the specification, the heterocycloalkyl group can be a monocyclic, bicyclic, tricyclic or tetracyclic ring system, which can include fused or bridged ring systems; and the nitrogen, carbon or sulfur atoms in the heterocycloalkyl group can be optionally oxidized; the nitrogen atom can be optionally quaternized; and the heterocycloalkyl group can be partially or fully saturated. By way of example, the term “C₄-C₇ heterocycloalkyl group” contains at least 4 but no more than 7 carbon atoms. The heterocycloalkyl group dioxyl (C₄H₇O₂—) represents a C₄ heterocycloalkyl group. The piperidyl group (C₅H₉N—) represents a C₅ heterocycloalkyl group.

As used herein, the term “therapeutically effective amount” refers to an amount (of a compound) that is sufficient to provide a therapeutic benefit to a patient in the treatment or management of a disease or disorder, or to delay or minimize one or more symptoms associated with the disease or disorder.

As used herein, the term “pharmaceutically acceptable salt” refers to any salt suitable for administration to a patient. Examples of salts include, but are not limited to, acid-derived, base-derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

In some embodiments, a compound having a formula (I) or a pharmaceutically acceptable salt thereof is presented

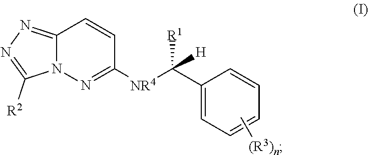

wherein
R¹ and R² are independently a C₁-C₆ alkyl group optionally substituted with one or more fluorine or a C₃-C₆ cycloalkyl group optionally substituted with one or more fluorine;
R³ is independently at each occurrence halogen, —CN, —OR⁵, —C(═O)R⁵, —NHC(═O)R⁵, —C(═O)NR⁵R⁶, —NR⁵R⁶, —P(═O)R⁵R⁶, a C₁-C₆ alkyl group optionally substituted with one or more fluorine, or a C₃-C₆ cycloalkyl group optionally substituted with one or more fluorine;
R⁴ is hydrogen or a C₁-C₆ alkyl group;
R⁵ and R⁶ are independently at each occurrence hydrogen, a C₁-C₆ alkyl group, or R⁵ and
R⁶ together with the nitrogen to which each is attached form a C₃-C₆ heterocycloalkyl group optionally substituted with one or more fluorine; and
“n” is an integer from 1 to 5.

In some embodiments, R¹ is a C₁-C₆ alkyl group. Non-limiting examples of a C₁-C₆ alkyl group include a methyl group, an ethyl group, an isopropyl group and the like. In some such embodiments, R⁴ is hydrogen, a methyl group, or an ethyl group.

In some embodiments, R² is a C₁-C₆ alkyl group substituted with one or more fluorine. Non-limiting examples of a C₁-C₆ alkyl group substituted with one or more fluorine include a CH₂F group, a CHF₂ group, or a CF₃ group. In some such embodiments, R⁴ is hydrogen, a methyl group, or an ethyl group.

In some embodiments, R¹ is a C₁-C₆ alkyl group, and R³ is independently at each occurrence halogen, —CN, —OR⁵, or a C₁-C₆ alkyl group optionally substituted with one or more fluorine. Non-limiting examples of a C₁-C₆ alkyl group include a methyl group, an ethyl group, an isopropyl group and the like. In some such embodiments, R⁴ is hydrogen, a methyl group, or an ethyl group.

In some embodiments, R² is a C₁-C₆ alkyl group substituted with one or more fluorine, and R³ is independently at each occurrence halogen, —CN, —OR⁵, or a C₁-C₆ alkyl group optionally substituted with one or more fluorine. Non-limiting examples of a C₁-C₆ alkyl group substituted with one or more fluorine include a CH₂F group, a CHF₂ group, or a CF₃ group. In some such embodiments, R⁴ is hydrogen, a methyl group, or an ethyl group.

In some embodiments, R¹ is a C₁-C₆ alkyl group, R² is a C₁-C₆ alkyl group substituted with one or more fluorine, and R³ is independently at each occurrence halogen, —CN, —OR⁵, or a C₁-C₆ alkyl group optionally substituted with one or more fluorine. Non-limiting examples of a C₁-C₆ alkyl group include a methyl group, an ethyl group, an isopropyl group and the like. Non-limiting examples of a C₁-C₆ alkyl group substituted with one or more fluorine include a CH₂F group, a CHF₂ group, or a CF₃ group. In some such embodiments, R⁴ is hydrogen, a methyl group, or an ethyl group.

In some embodiments, R¹ is a C₁-C₆ alkyl group, R² is a C₁-C₆ alkyl group substituted with one or more fluorine, and R³ is independently at each occurrence halogen. Non-limiting examples of a C₁-C₆ alkyl group include a methyl group, an ethyl group, an isopropyl group and the like. Non-limiting examples of a C₁-C₆ alkyl group substituted with one or more fluorine include a CH₂F group, a CHF₂ group, or a CF₃ group. In some such embodiments, R⁴ is hydrogen, a methyl group, or an ethyl group.

In some embodiments, a compound having a formula (II) or a pharmaceutically acceptable salt thereof is presented:

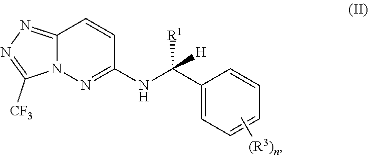

wherein R¹ is a C₁-C₆ alkyl group, R³ is independently at each occurrence halogen, —CN, —OR⁵, or a C₁-C₆ alkyl group optionally substituted with one or more fluorine, and “n” is an integer from 1 to 5.

In some embodiments, a compound having a formula (III) or a pharmaceutically acceptable salt thereof is presented

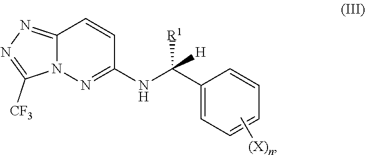

wherein R¹ is a C₁-C₆ alkyl group, X is halogen and “n” is an integer from 1 to 5.

In some embodiments, a compound having a formula (IV) or a pharmaceutically acceptable salt thereof is presented

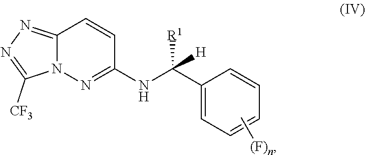

wherein R¹ is a C₁-C₆ alkyl group and “n” is an integer from 1 to 5.

In some embodiments, a compound having a formula (V) or a pharmaceutically acceptable salt thereof is presented

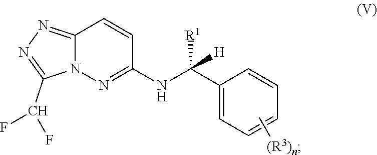

wherein R¹ is a C₁-C₆ alkyl group, R³ is independently at each occurrence halogen, —CN, OR⁵, or a C₁-C₆ alkyl group optionally substituted with one or more fluorine, and “n” is an integer from 1 to 5.

In some embodiments, a compound having a formula (VI) or a pharmaceutically acceptable salt thereof is presented

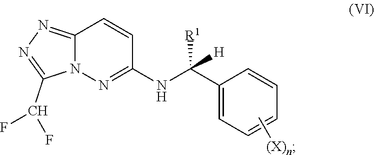

wherein R¹ is a C₁-C₆ alkyl group, X is halogen, and “n” is an integer from 1 to 5.

In some embodiments, a compound having a formula (VII) or a pharmaceutically acceptable salt thereof is presented

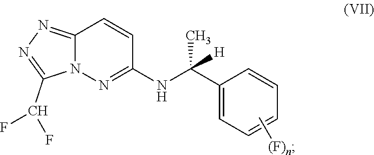

wherein R¹ is a C₁-C₆ alkyl group and “n” is an integer from 1 to 5.

Non-limiting examples of compounds according to embodiments of the present disclosure include:

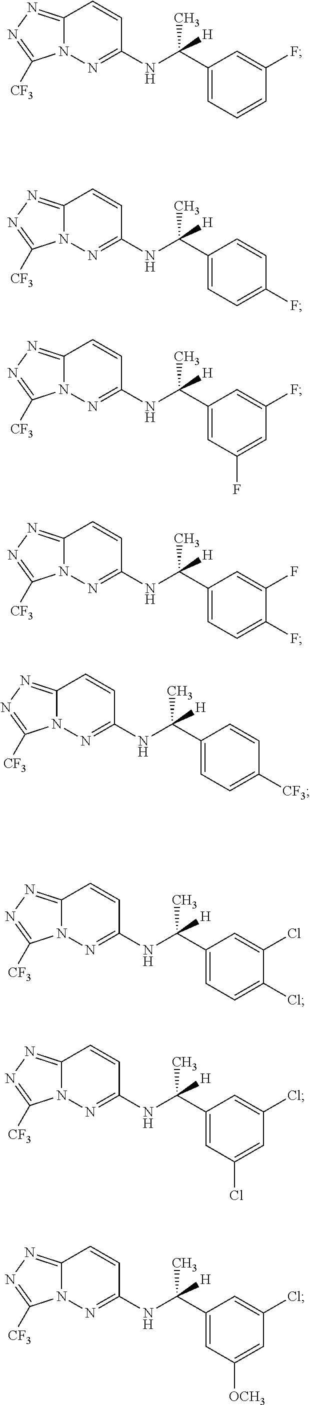

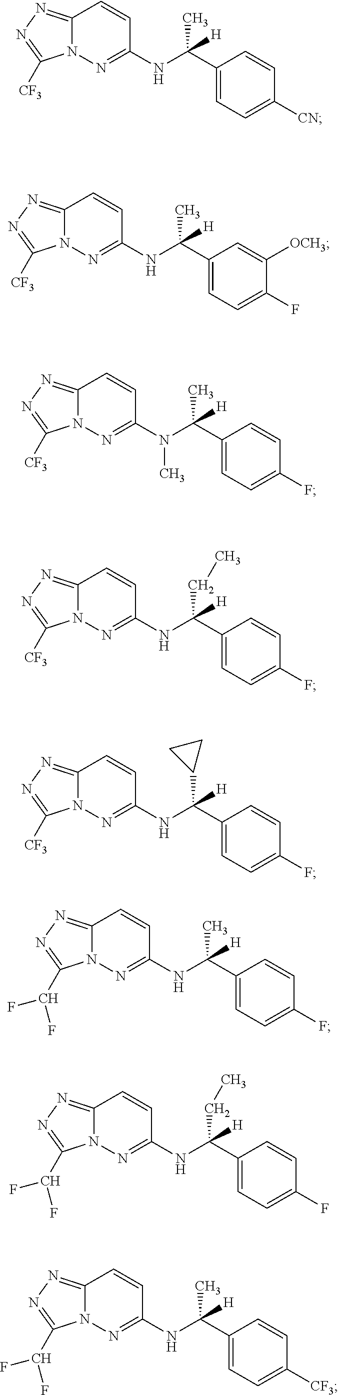

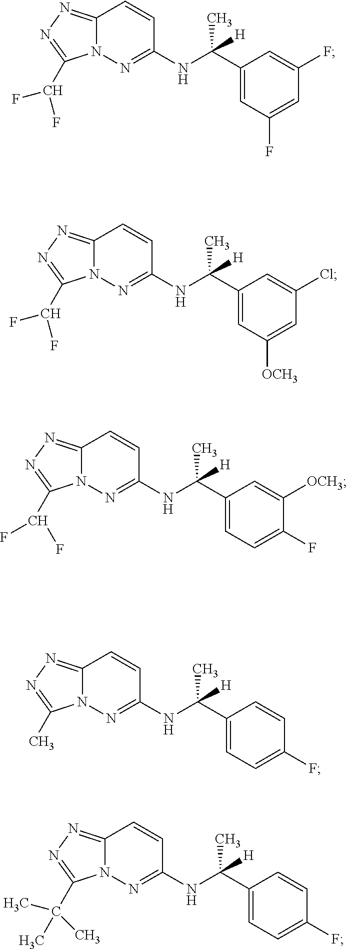 [SMILES: C[C@@H](Nc1ccc2nnc(C(F)F)n2n1)c1cc(F)cc(F)c1 ; COc1cc(Cl)cc([C@@H](C)Nc2ccc3nnc(C(F)F)n3n2)c1 ; COc1cc([C@@H](C)Nc2ccc3nnc(C(F)F)n3n2)ccc1F ; Cc1nnc2ccc(N[C@H](C)c3ccc(F)cc3)nn12 ; C[C@@H](Nc1ccc2nnc(C(C)(C)C)n2n1)c1ccc(F)cc1]

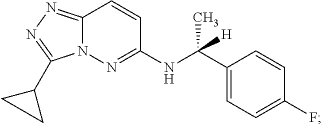 [SMILES: C[C@@H](Nc1ccc2nnc(C3CC3)n2n1)c1ccc(F)cc1]

or pharmaceutically acceptable salts thereof.

In accordance with embodiments of the present invention, compounds having formula (I) to (VII) are optically enriched R enantiomers. In accordance with embodiments of the present invention, compounds having formula (I) to (VII) are preferably R enantiomers. Without being bound by any theory it is believed that, in some embodiments, compounds of the present description may be capable of targeting NTD of AR. In some embodiments, R enantiomers of the present description may exhibit preferential targeting of NTD of AR as compared to corresponding S enantiomers or racemic mixtures. As noted earlier, targeting NTD of AR may facilitate inhibition of AR function and also mitigate the current drug resistance mechanisms that may develop post androgen-deprivation or LBD inhibitor treatment. Further, targeting NTD may mitigate variant and LBD point mutation driven resistance thereby providing effective treatment against all stages of AR driven prostate cancer.

In some embodiments, the compounds of the present description may be capable of targeting NTD of ARV7. ARV7 may include NTD, DBD and a cryptic exon. NTD of ARV7 may include Tau1, Tau5 and a FXXLF motif. In some embodiments, the compounds of the present description may be capable of targeting FXXLF motif of ARV7.

In some embodiments, a pharmaceutical composition is presented. The pharmaceutical composition includes a compound as described herein above or a pharmaceutically acceptable salt, solvate, hydrate, polymorph, or co-crystal thereof, and a pharmaceutically carrier, diluent, or excipient. In some embodiments, the pharmaceutical composition includes a compound having a formula (I) to (VII). The pharmaceutical composition may further include one or more other additional anticancer therapeutic agents in some embodiments.

The pharmaceutical compositions of the present invention may be in any form that allows for the composition to be administered to a subject. For example, the composition may be in the form of a solid, liquid or gas (aerosol). Pharmaceutical compositions may be formulated so as to allow the active ingredients contained therein to be bioavailable upon administration of the composition to a subject. To further optimise the pharmacokinetic profile of the compounds of the present invention, the compounds may be administered in conjunction with a suitable delivery vehicle (e.g., microcapsules, microspheres, biodegradable polymer films, lipid-based delivery systems such as liposomes and lipid foams, viscous instillates and absorbable mechanical barriers) useful for maintaining the necessary concentrations of the prodrugs or the therapeutic agent at the site of the disease.

A process for preparing a pharmaceutical composition is also presented. The process includes mixing a compound as described hereinabove or a pharmaceutically acceptable salt, solvate, hydrate, polymorph, or co-crystal thereof with a pharmaceutically acceptable carrier, diluent, or excipient. In some embodiments, the process includes mixing a compound having a formula (I) to (VII) or a pharmaceutically acceptable salt, solvate, hydrate, polymorph, or co-crystal thereof with a pharmaceutically acceptable carrier, diluent, or excipient.

In some embodiments, methods of treating or reducing symptoms of a certain disease by administering a compound of the present invention are also presented. The compounds or derivatives thereof can be administered to any host, including a human, a non-human animal and mammals, in an amount effective to treat a disorder.

In some embodiments, the compounds of the present description may be used as single agents or in combination with other additional anticancer therapeutic agents for treating metastatic castration-resistant prostate cancer, metastatic castration-sensitive prostate cancer, non-metastatic castration-resistant prostate cancer, non-metastatic castration-sensitive prostate cancer, localized prostate cancer or metastatic/non-metastatic castration resistant prostate cancer that exhibits intrinsic or acquired resistance to enzalutamide, abiraterone acetate or any other androgen signalling axis/receptor inhibitors.

In some embodiments, the compounds of the present description may be used as single agents or in combination with other additional anticancer therapeutic agents for treating AR positive HER2 positive metastatic, advanced breast cancer, AR positive advanced TNBC, ER negative HER2 positive (ER−HER2+) breast cancer, or ER positive HER2 negative (ER+HER2−) breast cancer that exhibit poor therapeutic efficacy against known ER antagonists.

In some embodiments, the compounds of the present description may be used as single agents or in combination with other additional anticancer therapeutic agents for treating ovarian cancer. In some embodiments, the compounds of the present description may be used as single agents or in combination with other additional anticancer therapeutic agents for treating BRAF/MEK targeted therapy resistant melanoma.

In some embodiments, the compounds of the present description may be used as single agents or in combination with other additional AR antagonists or AR LBD inhibitors. Non-limiting examples of other additional AR antagonists or AR LBD inhibitors include bicalutamide, enzalutamide, flutamide, nilutamide, apalutamide, darolutmide, proxalutamide, or combinations thereof.

In some embodiments, a method of treating prostate cancer, breast cancer ovarian cancer, or melanoma is presented. In some embodiments, a method of treating prostate cancer is presented. Non-limiting examples of prostate cancer include metastatic castration-resistant prostate cancer, metastatic castration-sensitive prostate cancer, non-metastatic castration-resistant prostate cancer, non-metastatic castration-sensitive prostate cancer, localized prostate cancer or metastatic/non-metastatic castration resistant prostate cancer that exhibits intrinsic or acquired resistance to enzalutamide, abiraterone acetate or any other androgen signalling axis/receptor inhibitors. Non-limiting examples of breast cancer include AR positive HER2 positive metastatic, advanced breast cancer, AR positive advanced TNBC, ER negative HER2 positive (ER−HER2+) breast cancer, and ER positive HER2 negative (ER+HER2−) breast cancer that exhibit poor therapeutic efficacy against known ER antagonists. Non-limiting example of melanoma includes BRAF/MEK targeted therapy resistant melanoma.

The method includes administering to a patient an effective amount of a pharmaceutical composition including a compound of the present invention or a pharmaceutically acceptable salt, solvate, hydrate, polymorph, or co-crystal thereof. The pharmaceutical composition may further one or more other additional anticancer therapeutic agents in some embodiments.

The pharmaceutical composition may be administered by any suitable method known to a person skilled in the art. Typical routes of administration include, without limitation, oral, topical, parenteral, sublingual, rectal, vaginal, ocular, and intranasal. The term parenteral as used herein includes intravenous, intraperitoneal, intramuscular, intradermal, and epidermal including subcutaneous and intradermal, oral, or application to mucosal surfaces, e.g, by intranasal administration using inhalation of aerosol suspensions, and by implanting to muscle or other tissue in the subject.

### SPECIFIC EMBODIMENTS

The following enumerated embodiments are representative of some aspects of the invention.
Embodiment 1. A compound having a formula (I) or a pharmaceutically acceptable salt thereof:

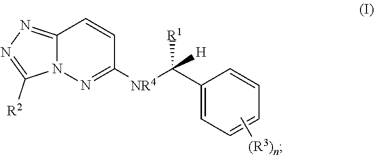

wherein
R¹ and R² are independently a C₁-C₆ alkyl group optionally substituted with one or more fluorine or a C₃-C₆ cycloalkyl group optionally substituted with one or more fluorine;
R³ is independently at each occurrence halogen, —CN, —OR⁵, —C(═O)R⁵, —NHC(═O)R⁵, —C(═O)NR⁵R⁶, —NR⁵R⁶, —P(═O)R⁵R⁶, a C₁-C₆ alkyl group optionally substituted with one or more fluorine, or a C₃-C₆ cycloalkyl group optionally substituted with one or more fluorine;
R⁴ is hydrogen or a C₁-C₆ alkyl group;
R⁵ and R⁶ are independently at each occurrence hydrogen, a C₁-C₆ alkyl group, or R⁵ and
R⁶ together with the nitrogen to which each is attached form a C₃-C₆ heterocycloalkyl group optionally substituted with one or more fluorine; and
“n” is an integer from 1 to 5.
Embodiment 2. The compound of embodiment 1, wherein R¹ is a C₁-C₆ alkyl group.
Embodiment 3. The compound of embodiment 1 or 2, wherein R² is a C₁-C₆ alkyl group substituted with one or more fluorine.
Embodiment 4. The compound of any one of embodiments 1-3, wherein R¹ is a C₁-C₆ alkyl group, and R³ is independently at each occurrence halogen, —CN, —OR⁴, or a C₁-C₆ alkyl group optionally substituted with one or more fluorine.
Embodiment 5. The compound of any one of embodiments 1-4, wherein R² is a C₁-C₆ alkyl group substituted with one or more fluorine, and R³ is independently at each occurrence halogen, —CN, —OR⁴, or a C₁-C₆ alkyl group optionally substituted with one or more fluorine.
Embodiment 6. The compound of any one of embodiments 1-5, wherein R¹ is a C₁-C₆ alkyl group, R² is a C₁-C₆ alkyl group substituted with one or more fluorine, and R³ is independently at each occurrence halogen, —CN, —OR⁴, or a C₁-C₆ alkyl group optionally substituted with one or more fluorine.
Embodiment 7. The compound of any one of embodiments 1-6, wherein R¹ is a C₁-C₆ alkyl group, R² is a C₁-C₆ alkyl group substituted with one or more fluorine, and R³ is independently at each occurrence halogen.
Embodiment 8. The compound of embodiment 1 having a formula (II) or a pharmaceutically acceptable salt thereof:

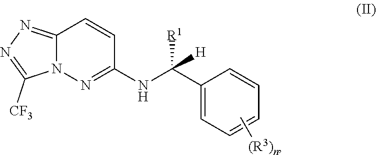

wherein R¹ is a C₁-C₆ alkyl group, R³ is independently at each occurrence halogen, —CN, —OR⁵, or a C₁-C₆ alkyl group optionally substituted with one or more fluorine, and “n” is an integer from 1 to 5.
Embodiment 9. The compound of embodiment 1 or 8 having a formula (II) or a pharmaceutically acceptable salt thereof:

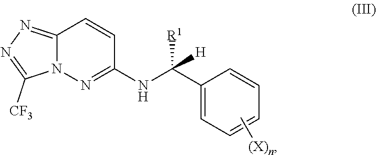

wherein R¹ is a C₁-C₆ alkyl group, X is halogen and “n” is an integer from 1 to 5.
Embodiment 10. The compound of any one of embodiments 1, 8 or 9 having a formula (IV) or a pharmaceutically acceptable salt thereof:

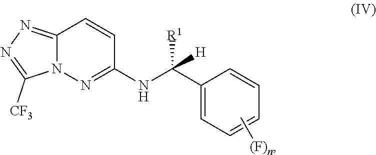

wherein R¹ is a C₁-C₆ alkyl group and “n” is an integer from 1 to 5.
Embodiment 11. The compound of embodiment 1 having a formula (V) or a pharmaceutically acceptable salt thereof:

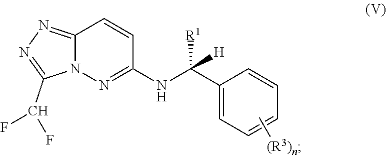

wherein R¹ is a C₁-C₆ alkyl group, R³ is independently at each occurrence halogen, —CN, —OR⁵, or a C₁-C₆ alkyl group optionally substituted with one or more fluorine, and “n” is an integer from 1 to 5.
Embodiment 12. The compound of embodiment 1 or 11 having a formula (VI) or a pharmaceutically acceptable salt thereof:

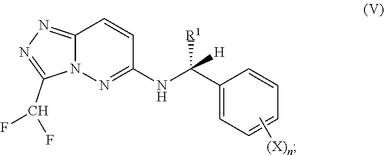

wherein R¹ is a C₁-C₆ alkyl group, X is halogen, and “n” is an integer from 1 to 5.
Embodiment 13. The compound of any one of embodiments 1, 11 or 12 having a formula (VII) or a pharmaceutically acceptable salt thereof:

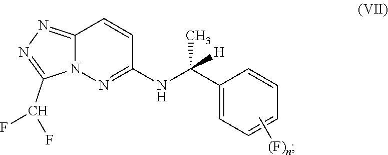

wherein R¹ is a C₁-C₆ alkyl group and “n” is an integer from 1 to 5.
Embodiment 14. The compound of any one of embodiments 1-13 selected from the group consisting of:

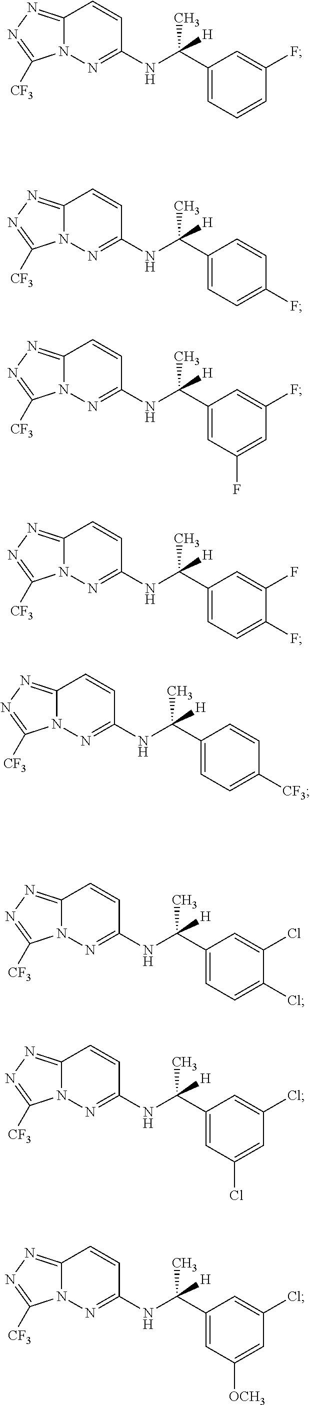

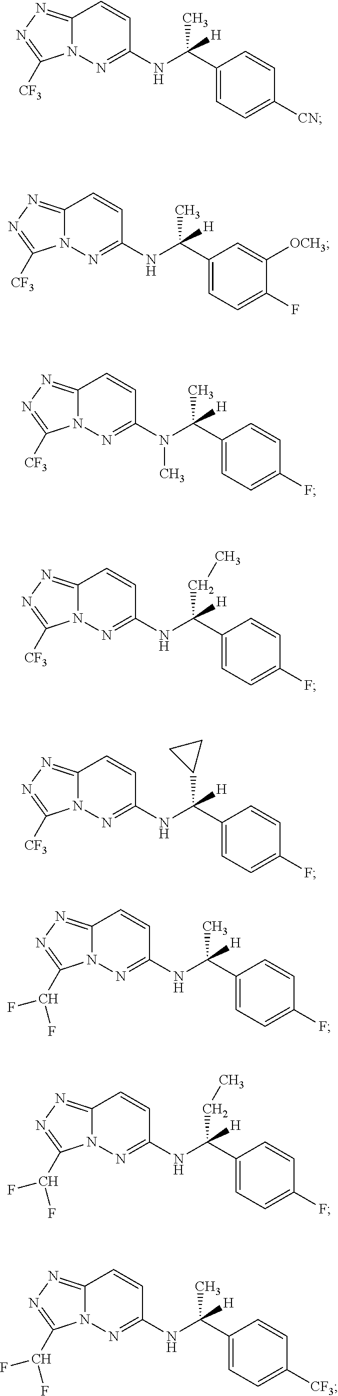

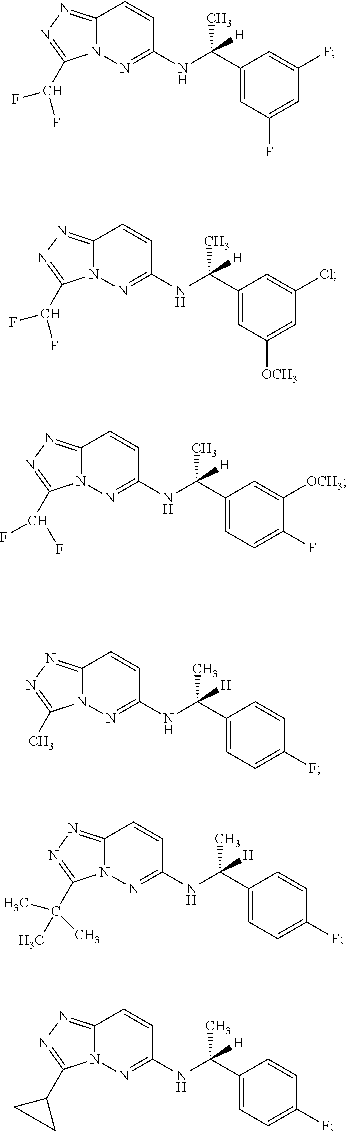 [SMILES: C[C@@H](Nc1ccc2nnc(C(F)F)n2n1)c1cc(F)cc(F)c1 ; COc1cc(Cl)cc([C@@H](C)Nc2ccc3nnc(C(F)F)n3n2)c1 ; COc1cc([C@@H](C)Nc2ccc3nnc(C(F)F)n3n2)ccc1F ; Cc1nnc2ccc(N[C@H](C)c3ccc(F)cc3)nn12 ; C[C@@H](Nc1ccc2nnc(C(C)(C)C)n2n1)c1ccc(F)cc1 ; C[C@@H](Nc1ccc2nnc(C3CC3)n2n1)c1ccc(F)cc1]

and any pharmaceutically acceptable salts thereof.
Embodiment 15. A pharmaceutical composition comprising the compound of any one of embodiments 1-14 or a pharmaceutically acceptable salt, solvate, hydrate, polymorph, or co-crystal thereof, and a pharmaceutically carrier, diluent, or excipient.
Embodiment 16. A pharmaceutical composition comprising a compound of embodiment 8 or 11 or a pharmaceutically acceptable salt, solvate, hydrate, polymorph, or co-crystal thereof; and a pharmaceutically carrier, diluent, or excipient.
Embodiment 17. A method for modulation of androgen receptor activity for treatment of prostate cancer, breast cancer, ovarian cancer, or melanoma, the method comprising administering to a patient a therapeutically effective amount of a pharmaceutical composition comprising a compound of any one of embodiments 1-14.
Embodiment 18. The method of embodiment 17, wherein the method comprises modulation of androgen receptor activity for treatment of metastatic castration-resistant prostate cancer.
Embodiment 19. The method of embodiment 17 or 18, wherein the method comprises modulation of N-terminal domain of an androgen receptor for treatment of metastatic castration-resistant prostate cancer.
Embodiment 20. The method of any one of embodiments 17-19, wherein the method comprises administering to a patient a therapeutically effective amount of a pharmaceutical composition comprising a compound of any one of embodiments 1-14.

### EXAMPLES

### General Methods for Synthesis

### Example 1: Synthesis of 6-chloro-3-trifluoromethyl-[1,2,4]triazolo[4,3-b]pyridazine

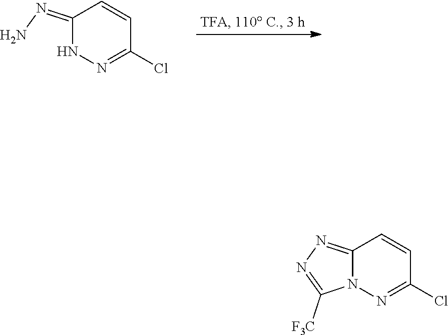 [SMILES: N/N=c1/ccc(Cl)n[nH]1 ; FC(F)(F)c1nnc2ccc(Cl)nn12]

In a sealed tube trifluoroacetic acid (60 mL) was added followed by 3-chloro-6-hydrazinopyridazine (11.0 g, 0.049 mol, 1 eq). The reaction mixture was heated at 110° C. for 3 h. The reaction mixture was evaporated to dryness. The crude mass was dissolved in ethyl acetate and washed with sodium bicarbonate aqueous solution three times. The organic layer was separated, and the aqueous layer was extracted with ethyl acetate twice. The combined organic layers were washed with brine, dried over sodium sulphate, filtered, and concentrated under reduced pressure to obtain 6-chloro-3-trifluoromethyl-[1,2,4]triazolo[4,3-b]pyridazine as an off white solid (11.5 g; 35% yield). 1H NMR (400 MHz, chloroform-d) δ 8.23 (d, J=8.0 Hz, 1H), 7.35 (d, J=8.0 Hz, 1H).

### Example 2 Synthesis of (R, S)—N-(1-(4-fluorophenyl)ethyl)-3-(trifluoromethyl)-[1,2,4]triazolo[4,3-b]pyridazin-6-amine (Comparative Compound 1)

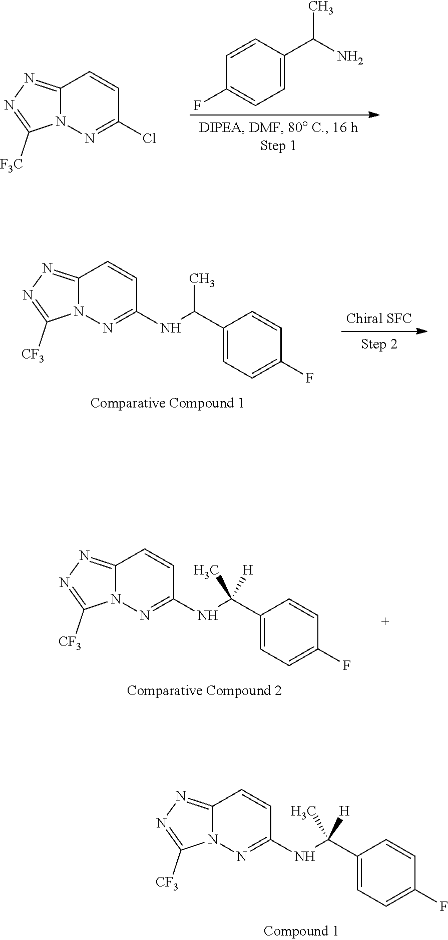

### Step 1: Synthesis of (R, S)—N-(1-(4-fluorophenyl)ethyl)-3-(trifluoromethyl)-[1,2,4]triazolo[4,3-b]pyridazin-6-amine (Comparative Compound 1)

A stirred solution of 6-chloro-3-(trifluoromethyl)-[1,2,4]triazolo[4,3-b]pyridazine (1 g, 4.5 mmol), (R,S)-1-(4-fluorophenyl) ethan-1-amine (0.75 g, 5.4 mmol) and DIPEA (2.4 mL, 13.5 mmol) in DMF (10 mL) was heated to 80° C. After 16 h the reaction mixture was cooled to 25° C.-30° C., ice cold water was added, and the organic product was extracted into EtOAc. The EtOAc layer was dried over anhydrous sodium sulphate and concentrated under reduced pressure to obtain a crude product which was purified by silica gel column chromatography to give (R,S)—N-(1-(4-fluorophenyl)ethyl)-3-(trifluoromethyl)-[1,2,4]triazolo[4,3-b]pyridazin-6-amine as a white solid (0.7 g; 72% yield). LCMS: (m/z=326.29 [M+H] +). HPLC: 99.43%

1H-NMR (400 MHz, DMSO-d6): δ 8.27 (d, J=7.20 Hz, 1H), 8.09 (d, J=10.0 Hz, 1H), 7.43-7.40 (m, 2H), 7.15 (t, J=8.80 Hz, 2H), 7.02 (d, J=10.0 Hz, 1H), 4.87-4.80 (m, 1H), 1.50 (d, J=6.80 Hz, 3H).

### Step 2: Separation of Comparative Compound 1 into (R)—N-(1-(4-fluorophenyl)ethyl)-3-(trifluoromethyl)-[1,2,4]triazolo[4,3-b]pyridazin-6-amine (Compound 1) and(S)—N-(1-(4-fluorophenyl)ethyl)-3-(trifluoromethyl)-[1,2,4]triazolo[4,3-b]pyridazin-6-amine (Comparative Compound 2)

The product of step 1 (108 mg) was separated into the two enantiomers by Chiral SFC {Column/dimensions: (R,R) Whelk-O1 (30*250) mm, 5μ; % CO2: 80%; % Co solvent: 20% (ACN: IPA) (1:1); Flow: 100 g/min; Back Pressure: 100 bar; UV: 235 nm}.

The slower moving component, (longer retention time) gave (R)—N-(1-(4-fluorophenyl)ethyl)-3-(trifluoromethyl)-[1,2,4]triazolo[4,3-b]pyridazin-6-amine (Compound 1), as an off-white solid (40 mg). LCMS: (m/z=326.39 [M+H] +), HPLC: 99.45%, Specific Optical Rotation: +286.2° (c, 0.1%, MeOH)

1HNMR 400 MHz, DMSO-d6: δ 8.27 (d, J=6.80 Hz, 1H), 8.08 (d, J=10.0 Hz, 1H), 7.43-7.40 (m, 2H), 7.14 (t, J=8.80 Hz, 2H), 7.03 (d, J=10.0 Hz, 1H), 4.87-4.80 (m, 1H), 1.50 (d, J=6.80 Hz, 3H).

The faster moving component, (shorter retention time) gave(S)—N-(1-(4-fluorophenyl)ethyl)-3-(trifluoromethyl)-[1,2,4]triazolo[4,3-b]pyridazin-6-amine (Comparative Compound 2), as an off-white solid (40 mg). LCMS: (m/z=326.29 [M+H] +), HPLC: 99.07%; Specific Optical Rotation: −291.2° (c, 0.1%, MeOH)

1HNMR 400 MHz, DMSO-d6: δ 8.26 (d, J=6.80 Hz, 1H), 8.08 (d, J=10.0 Hz, 1H), 7.43-7.40 (m, 2H), 7.14 (t, J=8.80 Hz, 2H), 7.02 (d, J=10.0 Hz, 1H), 4.87-4.80 (m, 1H), 1.50 (d, J=6.80 Hz, 3H).

### Example 3: Synthesis of (R)—N-(1-(4-fluorophenyl)ethyl)-3-(trifluoromethyl)-[1,2,4]triazolo[4,3-b]pyridazin-6-amine (Compound 1)

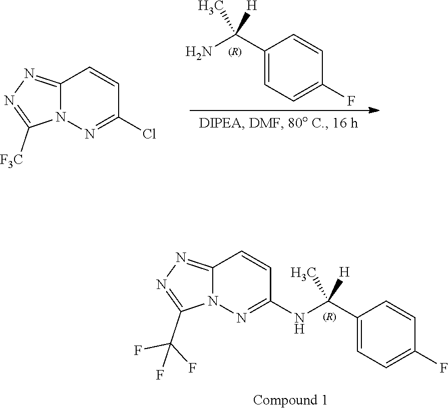

A stirred solution of 6-chloro-3-(trifluoromethyl)-[1,2,4]triazolo[4,3-b]pyridazine (0.8 g, 3.59 mmol), (R)-1-(4-fluorophenyl) ethan-1-amine {Combi Blocks, Cat No: SS-7617, Lot NO: B46320; Specific Optical Rotation: +23.3° (c 1% in MeOH)} (0.59 g, 4.3 mmol) and DIPEA (1.9 mL, 10.8 mmol) in DMF (8 mL) was heated to 80° C. After 16 h the reaction mixture was cooled to 25° C.-30° C., ice cold water was added, and the organic product was extracted into EtOAc. The EtOAc layer was dried over anhydrous sodium sulphate and concentrated under reduced pressure to obtain a crude product which was purified by silica gel column chromatography to give (R)—N-(1-(4-fluorophenyl)ethyl)-3-(trifluoromethyl)-[1,2,4]triazolo[4,3-b]pyridazin-6-amine as a white solid (0.7 g; 72% yield). LCMS: (m/z=326.29 [M+H] +; HPLC: 97.33%; Specific Optical Rotation: +291° (c 0.1% in MeOH)

1HNMR (400 MHz, DMSO-d6): δ 8.26 (d, J=6.80 Hz, 1H), 8.08 (d, J=10.0 Hz, 1H), 7.43-7.40 (m, 2H), 7.14 (t, J=8.80 Hz, 2H), 7.02 (d, J=10.0 Hz, 1H), 4.85-4.84 (m, 1H), 1.50 (d, J=6.80 Hz, 3H).

### Example 4: Synthesis of(S)—N-(1-(4-fluorophenyl)ethyl)-3-(trifluoromethyl)-[1,2,4]triazolo[4,3-b]pyridazin-6-amine (Comparative Compound 2)

Comparative compound 2 was synthesized using the procedure described in Example 3 by using(S)-1-(4-fluorophenyl) ethan-1-amine {BLD Pharmatech, Cat No: BD47207, Lot NO: CKA477; Specific Optical Rotation: −24.1° (c 1% in MeOH)} as a starting material. (S)—N-(1-(4-fluorophenyl)ethyl)-3-(trifluoromethyl)-[1,2,4]triazolo[4,3-b] pyridazin-6-amine was obtained as a white solid (1.3 g, 90% yield). LCMS: (m/z=326.29 [M+H] +); HPLC: 99.56%; Specific Optical Rotation: −305.6° (c 0.1% in MeOH)

1HNMR (400 MHz, DMSO-d6): δ 8.26 (d, J=6.80 Hz, 1H), 8.08 (d, J=10.00 Hz, 1H), 7.40-7.41 (m, 2H), 7.14 (d, J=8.80 Hz, 2H), 7.02 (d, J=10.00 Hz, 1H), 4.82-4.80 (m, 1H), 1.50 (d, J=6.80 Hz, 3H).

### Example 5: Synthesis of (R)—N-(1-(3,5-difluorophenyl)ethyl)-3-(trifluoromethyl)-[1,2,4]triazolo[4,3-b]pyridazin-6-amine (Compound 2)

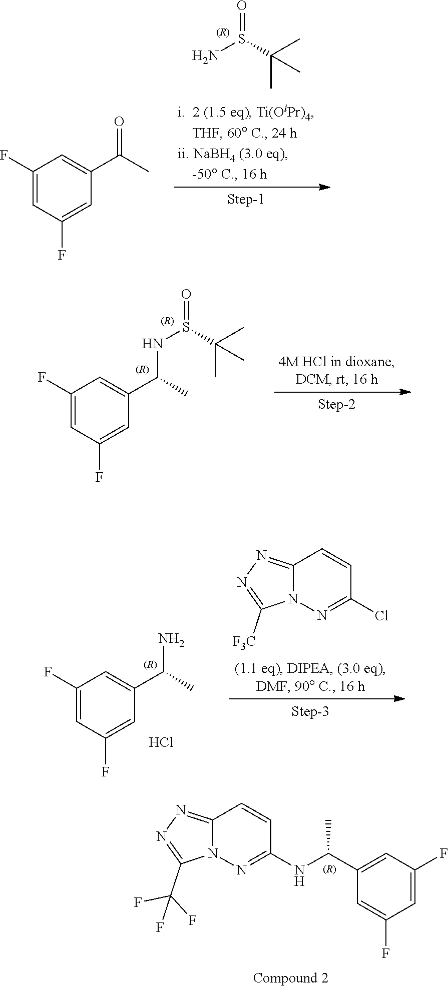

### Step-1: Synthesis of (R)—N—((R)-1-(3,5-difluorophenyl)ethyl)-2-methylpropane-2-sulfinamide

To the solution of 1-(3,5-difluorophenyl) ethanone (2.0 g, 0.0132 mol, 1.0 eq) and (R)-2-methylpropane-2-sulfinamide (2.52 g, 0.0208 mol, 2.0 eq) in anhydrous THF (20 ml) at 0° C., was added titanium tetraisopropoxide (0.446 ml, 0.0262, 1.5 eq). The reaction was heated at 60° C. for 24 h. The reaction was cooled to −50° C. and then added sodium borohydride (0.486 g, 0.0132 mol, 3.0 eq) and allowed to stir 16 h at room temperature. The reaction was poured over crushed ice and extracted with ethyl acetate. The combined organic layer was washed with brine, dried over sodium sulphate, filtered, and concentrated. The residue was purified by silica gel chromatography, eluting with a gradient of 0 to 50% ethyl acetate in hexanes providing (R)—N—((R)-1-(3,5-difluorophenyl)ethyl)-2-methylpropane-2-sulfinamide as colourless liquid (1.2 g; 44% yield).

1HNMR (400 MHz, DMSO-d6): δ 7.17-7.13 (m, 2H), 7.10-7.05 (m, 1H), 5.80 (d, J=8 Hz, 1H), 4.42-4.39 (m, 1H), 1.38 (d, J=8 Hz, 3H), 1.12 (s, 9H).

### Step-2: Synthesis of (R)-1-(3,5-difluorophenyl) ethanamine hydrochloride

To the solution of (R)—N—((R)-1-(3,5-difluorophenyl)ethyl)-2-methylpropane-2-sulfinamide (1.2 g, 0.0046 mol, 1.0 eq) in DCM (20 ml) in a reaction vial was added 4M HCl in dioxane (15 ml) at 0° C. and stirred at room temperature for 16 h. The reaction was concentrated and co-precipitated with methanol: ether as co-solvent to provide (R)-1-(4-fluorophenyl) propan-1-amine hydrochloride as a white solid (0.75 g; 96%).

### Step-3: Synthesis (R)—N-(1-(3,5-difluorophenyl)ethyl)-3-(trifluoromethyl)-[1,2,4]triazolo[4,3-b]pyridazin-6-amine

To the solution of (R)-1-(3,5-difluorophenyl) ethanamine hydrochloride (0.350 g, 0.002 mol, 1 eq) in DMF (10 ml) in a reaction vial was added N, N-diisopropylethylamine (0.885 ml, 0.0069 mol, 3.0 eq) followed 6-chloro-3-(trifluoromethyl)-[1,2,4]triazolo[4,3-b]pyridazine (0.558 g, 0.002 mol, 1.1 eq). The reaction was sealed and heated at 90° C. for 16 h. The reaction was diluted with ethyl acetate, washed with brine, dried over sodium sulphate, filtered and concentrated. The residue was purified by silica gel chromatography, eluting with a gradient of 0 to 50% ethyl acetate in hexanes providing (R)—N-(1-(3,5-difluorophenyl)ethyl)-3-(trifluoromethyl)-[1,2,4]triazolo[4,3-b]pyridazin-6-amine as a white solid (0.07 g; 9%). LCMS: (m/z=344.28 [M+H] +); LCMS purity: 95.67%, Specific Optical Rotation: +264.16 (c 0.1% in MeOH).

1HNMR 400 MHz, DMSO-d6: δ (ppm) 8.28 (d, J=6.4 Hz, 1H), 8.10 (d, J=10 Hz, 1H), 7.09-7.02 (m, 4H), 7.02 (s, 1H), 4.85-4.79 (m, 1H), 1.50 (d, J=6.8 Hz, 3H).

### Example 6: Synthesis of (R)—N-(1-(4-fluorophenyl) propyl)-3-(trifluoromethyl)-[1,2,4]triazolo[4,3-b]pyridazin-6-amine (Compound 3)

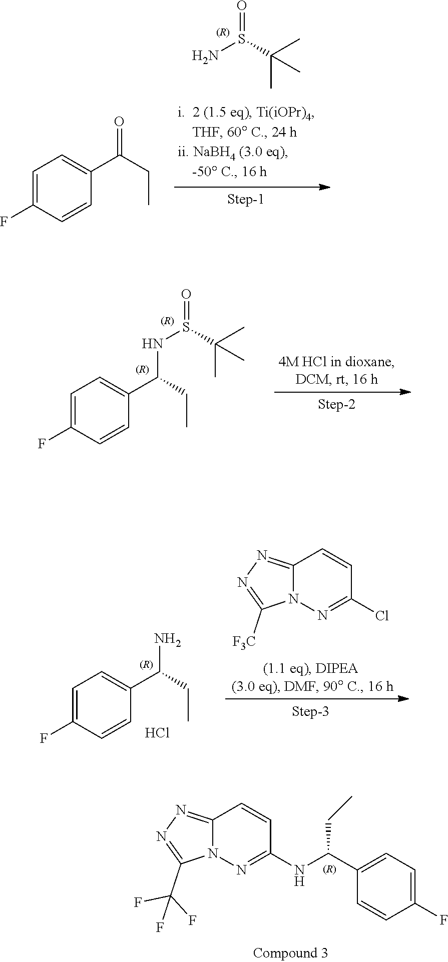

### Step-1: Synthesis of (R)—N—((R)-1-(4-fluorophenyl) propyl)-2-methylpropane-2-sulfinamide

To the solution of 1-(4-fluorophenyl) propan-1-one (2.0 g, 0.0132 mol, 1.0 eq) and (R)-2-methylpropane-2-sulfinamide (4.49 g, 0.0197 mol, 2.0 eq) in anhydrous tetrahydrofuran (20 ml) at 0° C., was added titanium tetraisopropoxide (0.318 ml, 0.0262, 1.5 eq). The reaction was heated at 60° C. for 24 h. The reaction was cooled to −50° C. and sodium borohydride (0.486 g, 0.0132 mol, 3.0 eq) was added and allowed to stir 16 h at room temperature. The reaction was poured over crushed ice and extracted with ethyl acetate. The combined organic layer was washed with brine, dried over sodium sulphate, filtered, and concentrated. The residue was purified by silica gel chromatography, eluting with a gradient of 0 to 50% ethyl acetate in hexanes providing (R)—N—((R)-1-(4-fluorophenyl) propyl)-2-methylpropane-2-sulfinamide as a colourless liquid (0.9 g; 26% yield). 1HNMR 400 MHz, DMSO-d6: δ (ppm) 7.41-7.38 (m, 2H), 7.13 (t, J=16 Hz, 2H), 5.55 (d, J=8 Hz 1H), 4.18-4.04 (m, 1H), 1.80-1.61 (m, 2H), 1.11 (s, 9H), 0.81 (t, J=8 Hz, 3H).

### Step-2: Synthesis of (R)-1-(4-fluorophenyl) propan-1-amine hydrochloride

To the solution of (R)—N—((R)-1-(4-fluorophenyl) propyl)-2-methylpropane-2-sulfinamide (0.9 g, 0.0035 mol, 1.0 eq) in DCM (20 ml) in a reaction vial was added 4M HCl in dioxane (15 ml) at 0° C. and stirred at room temperature for 16 h. The reaction was concentrated and co-precipitated with methanol: ether as co-solvent to provide (R)-1-(4-fluorophenyl) propan-1-amine hydrochloride as a white solid (0.5 g; 93% yield).

### Step-3: Synthesis of (R)—N-(1-(4-fluorophenyl) propyl)-3-(trifluoromethyl)-[1,2,4]triazolo[4,3-b]pyridazin-6-amine

To the solution of (R)-1-(4-fluorophenyl) propane-1-amine hydrochloride (0.350 g, 0.002 mol, 1 eq) in DMF (10 ml) was added diisopropylethylamine (0.885 ml, 0.0069 mol, 3.0 eq) followed by 6-chloro-3-(trifluoromethyl)-[1,2,4]triazolo[4,3-b] pyridazine (0.558 g, 0.002 mol, 1.1 eq) and then heated at 90° C. for 16 h. The reaction mixture was diluted with ethyl acetate, washed with brine, dried over sodium sulphate, filtered, and concentrated. The residue was purified by silica gel chromatography, eluting with a gradient of 0 to 50% ethyl acetate in hexanes providing (R)—N-(1-(4-fluorophenyl) propyl)-3-(trifluoromethyl)-[1,2,4]triazolo[4,3-b]pyridazin-6-amine as a white solid (0.08 g; 6% yield). LCMS: (m/z=340.51 [M+H] +); LCMS purity: 97.29%; Specific Optical Rotation: +283.66 (c 0.1% in MeOH).

1HNMR 400 MHz, DMSO-d6: δ (ppm) 8.24 (d, J=7.2 Hz, 1H), 8.07 (d, J=9.6 Hz, 1H), 7.39-7.36 (m, 2H), 7.13 (t, J=8.8 Hz, 2H), 7.04 (d, J=10 Hz, 1H), 4.57 (dd, J=14.4 Hz, 7.2 Hz, 1H), 1.94-1.87 (m, 1H), 1.80-1.73 (m, 1H), 0.88 (t, J=7.2 Hz, 3H).

### Example 7 Synthesis of (R)-3-(trifluoromethyl)-N-(1-(4-(trifluoromethyl)phenyl)ethyl)-[1,2,4]triazolo[4,3-b]pyridazin-6-amine (Compound 4)

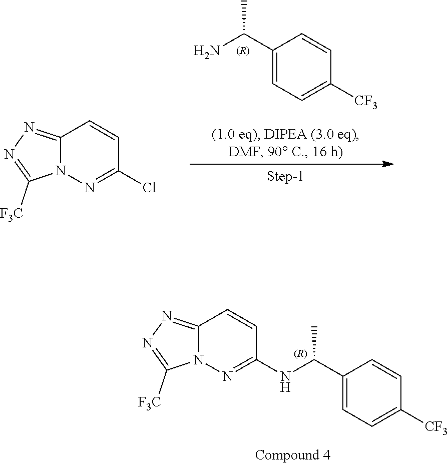

### Step-1: Synthesis of (R)-3-(trifluoromethyl)-N-(1-(4-(trifluoromethyl)phenyl)ethyl)-[1,2,4]triazolo[4,3-b]pyridazin-6-amine

To the solution of (R)-1-(4-(trifluoromethyl)phenyl) ethanamine (0.250 g, 0.00132 mol, 1 eq) in DMF (10 ml) was added N,N-diisopropylethylamine (0.510 ml, 0.00396 mol, 3.0 eq) followed 6-chloro-3-(trifluoromethyl)-[1,2,4]triazolo[4,3-b]pyridazine (0.323 g, 0.0014 mol, 1.1 eq). Reaction was heated in a sealed tube at 90° C. for 16 h. The reaction was diluted with ethyl acetate, washed with brine, dried over sodium sulphate, filtered, and concentrated. The residue was purified by silica gel chromatography, eluting with a gradient of 0 to 5% MeOH in dichloromethane providing (R)-3-(trifluoromethyl)-N-(1-(4-(trifluoromethyl)phenyl)ethyl)-[1,2,4]triazolo[4,3-b]pyridazin-6-amine as off white solid (0.230 g; 46% yield). LCMS: (m/z=376.41 [M+H] +); LCMS purity: 96.38%; Specific Optical Rotation: +186.60 (c 0.1% in MeOH)

1HNMR 400 MHz, DMSO-d6: δ 8.37 (d, J=6.8 Hz, 1H), 8.10 (d, J=10.0 Hz, 1H), 7.68 (d, J=8.4 Hz, 2H), 7.59 (d, J=8.4 Hz, 2H), 7.05 (d, J=10.0 Hz, 1H), 4.93-4.86 (m, 1H), 1.53 (d, J=6.8 Hz, 3H).

### Example 8: Synthesis of (R)—N-(1-(4-fluorophenyl)ethyl)-N-methyl-3-(trifluoromethyl)-[1,2,4]triazolo[4,3-b]pyridazin-6-amine (Compound 5)

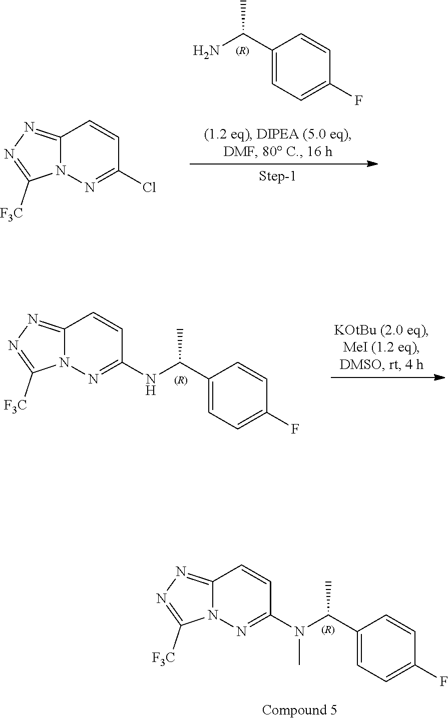

### Step-1: Synthesis of (R)—N-(1-(4-fluorophenyl)ethyl)-3-(trifluoromethyl)-[1,2,4]triazolo[4,3-b]pyridazin-6-amine

To the solution of (6-chloro-3-trifluoromethyl-[1,2,4]triazolo[4,3-b] pyridazine) (1.0 g, 0.004 mol, 1.0 eq) in DMF (20 ml) in a reaction vial was added N,N-diisopropylethylamine (8.14 ml, 0.013 m, 3.0 eq) followed by (R)-1-(4-fluorophenyl) ethan-1-amine (0.75 g, 0.005 mol, 1.2 eq). Reaction was sealed and heated at 80° C. for 16 h. The reaction was diluted with ethyl acetateand washed with brine, dried over sodium sulphate, filtered, and concentrated. The residue was purified by silica gel chromatography, eluting with a gradient of 0 to 70% ethyl acetate in hexanes providing (R)—N-(1-(4-fluorophenyl)ethyl)-3-(trifluoromethyl)-[1,2,4]triazolo[4,3-b]pyridazin-6-amine as a white solid (0.7 g, 22% yield). 1HNMR (400 MHz, DMSO-d6): δ 8.26 (d, J=6.80 Hz, 1H), 8.08 (d, J=10.0 Hz, 1H), 7.43-7.40 (m, 2H), 7.14 (t, J=8.80 Hz, 2H), 7.02 (d, J=10.0 Hz, 1H), 4.85-4.84 (m, 1H), 1.50 (d, J=6.80 Hz, 3H).

### Step-2: Synthesis of (R)—N-(1-(4-fluorophenyl)ethyl)-N-methyl-3-(trifluoromethyl)-[1,2,4]triazolo[4,3-b]pyridazin-6-amine

To the solution of (R)—N-(1-(4-fluorophenyl)ethyl)-3-(trifluoromethyl)-[1,2,4]triazolo[4,3-b]pyridazin-6-amine (0.4 g, 0.001 mol, 1.0 eq) in DMSO (10 ml) in a reaction vial was added potassium tert-butoxide (0.27 g, 0.024 mol, 2.0 eq) and stirred for 10 min at 0° C. Methyl iodide (0.2 g, 0.0014, 1.2 eq) was added and reaction was stirred for 4 h at room temperature. The reaction was quenched with water, extracted with dichloromethane twice. The combined organic layers were washed with brine, dried over sodium sulphate, filtered and concentrated. The residue was purified by silica gel chromatography, eluting with a gradient of 30% ethyl acetate in hexanes providing (R)—N,2-diphenyl-2-((3-(trifluoromethyl)-[1,2,4]triazolo[4,3-b]pyridazin-6-yl)amino) acetamide as a cream color solid (0.25 g, 60% yield). LCMS: (m/z=339.55 [M+H] +); HPLC: 97.37%; Specific Optical Rotation: +127.20 (c 0.1% in MeOH)

1HNMR 400 MHz, DMSO-d6: δ 8.27 (d, J=10.4 Hz, 1H), 7.58 (d, J=10.4 Hz, 1H), 7.43-7.40 (m, 2H), 7.22-7.16 (m, 2H), 5.72-5.67 (m, 1H), 2.86 (s, 3H), 1.59 (d, J=7.2 Hz, 3H).

### Example 9: Synthesis of (R)—N-(1-(4-fluoro-3-methoxyphenyl)ethyl)-3-(trifluoromethyl)-[1,2,4]triazolo[4,3-b]pyridazin-6-amine (Compound 6)

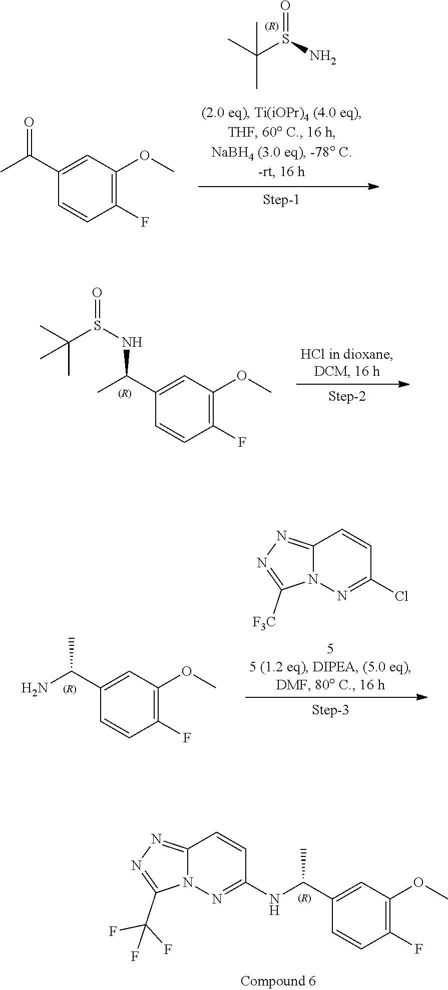

### Step-1: Synthesis of N—((R)-1-(4-fluoro-3-methoxyphenyl)ethyl)-2-methylpropane-2-sulfinamide

To the solution of 1-(4-fluoro-3-methoxyphenyl) ethan-1-one (0.5 g, 0.0029 mol, 1.0 eq) and (R)-2-methylpropane-2-sulfinamide (0.54 g, 0.0058 mol, 2.0 eq) in anhydrous tetrahydrofuran (20 ml) at 0° C., was added titanium tetraisopropoxide (3.4 mL, 0.011 mol, 4 eq). The reaction was heated at 60° C. for 16 h. The reaction was cooled to −50° C. and sodium borohydride (0.34 g, 0.012 mol, 3.0 eq) was added and allowed to stir overnight at room temp. The reaction was poured over crushed ice and extracted with ethyl acetate. The combined organic layer was washed with brine, dried over sodium sulphate, filtered, and concentrated. The residue was purified by silica gel chromatography, eluting with a gradient of 0 to 50% ethyl acetate in hexanes providing N—((R)-1-(4-fluoro-3-methoxyphenyl)ethyl)-2-methylpropane-2-sulfinamide as white solid (0.34 g, 44% yield). 1H NMR (400 MHz, CDCl3): δ 7.03-6.96 (m, 2H), 6.89-6.84 (m, 1H), 4.53-4.47 (m, 1H), 3.89 (d, J=4 Hz, 3H), 1.27 (s, 3H), 1.24 (s, 9H).

### Step-2: Synthesis of (R)-1-(4-fluoro-3-methoxyphenyl) ethan-1-amine hydrochloride

To the solution of N—((R)-1-(4-fluoro-3-methoxyphenyl)ethyl)-2-methylpropane-2-sulfinamide (0.3 g, 0.001 mol, 1.0 eq) in dichloromethane (20 ml) in a reaction vial was added 4M HCl in dioxane (5 ml) at 0° C. and stirred at room temperature for overnight. The reaction was concentrated and co-precipitated with MeOH: ether as co-solvent to provide (R)-1-(4-fluoro-3-methoxyphenyl) ethan-1-amine hydrochloride as white solid (0.2 g, 22% yield). HNMR 400 MHz, DMSO-d6: δ 8.38 (s, 3H), 7.41 (d, J=8 Hz, 1H), 7.25 (d, J=20 Hz, 1H), 7.04 (d, J=4 Hz, 1H), 4.42-4.36 (m, 1H), 3.86 (s, 3H), 1.49 (d, J=8 Hz, 3H).

### Step-3: Synthesis of (R)—N-(1-(4-fluoro-3-methoxyphenyl)ethyl)-3-(trifluoromethyl)-[1,2,4]triazolo[4,3-b]pyridazin-6-amine

To the solution of 6-chloro-3-(trifluoromethyl)-[1,2,4]triazolo[4,3-b] pyridazine (0.26 g, 0.001 mol, 1.2 eq) in DMF (10 ml) in a reaction vial was added N,N-diisopropylethylamine (0.47 ml, 0.002 mol, 5.0 eq) followed by (R)-1-(4-fluoro-3-methoxyphenyl) ethan-1-amine hydrochloride (0.2 g, 0.001 mol, 1.0 eq). Reaction was sealed and heated at 80° C. for 16 h. The reaction was diluted with ethyl acetate and washed with brine, dried over sodium sulphate, filtered and concentrated. The residue was purified by silica gel chromatography, eluting with a gradient of 0 to 70% ethyl acetate in hexanes providing (R)—N-(1-(4-fluoro-3-methoxyphenyl)ethyl)-3-(trifluoromethyl)-[1,2,4]triazolo[4,3-b]pyridazin-6-amine as a white solid (0.1 g, 38% yield). LCMS: (m/z=354.7 [M−H]+); HPLC: 97.65%; Specific Optical Rotation: +201.98 (c 0.1% in MeOH)

1NMR: HNMR 400 MHz, DMSO-d6: δ 8.22 (d, J=6.8 Hz, 1H), 8.08 (d, J=10.0 Hz, 1H), 7.18-7.12 (m, 2H), 7.02 (d, J=10 Hz, 1H), 6.95-6.92 (m, 1H), 4.84-4.78 (m, 1H), 3.83 (s, 3H), 1.50 (d, J=6.8 Hz, 3H).

### Example 10: Synthesis of (R)—N-(1-(3-chloro-5-methoxyphenyl)ethyl)-3-(trifluoromethyl)-[1,2,4]triazolo[4,3-b]pyridazin-6-amine (Compound 7)

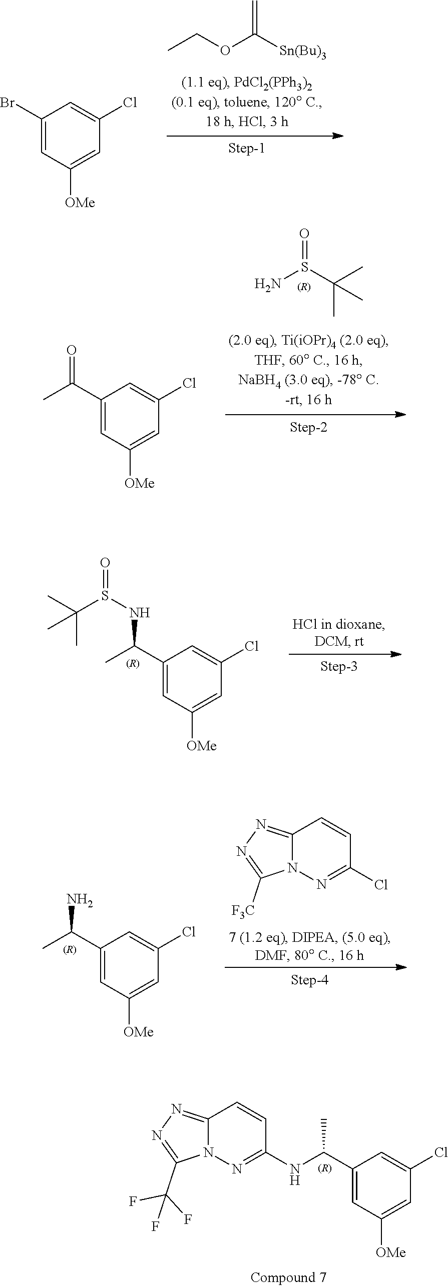

### Step-1: Synthesis of 1-(3-chloro-5-methoxyphenyl) ethan-1-one2

To the mixture of 1-bromo-3-chloro-5-methoxybenzene (5.0 g, 0.022 mol, 1 eq) and tributyl(1-ethoxyvinyl) stannane (9.05 g, 0.025 mol, 1.1 eq) in a sealed tube was added toluene (50 ml). Reaction was degassed with nitrogen for 10 min followed by addition of PdCl2(PPh3)2 (1.59 g, 0.002 mol, 0.1 eq). The reaction mixture was heated at 120° C. for 18 h. The reaction mixture was cooled to 0° C. and concentrated HCl (7.5 ml) was added and stirred for 3 h. The aqueous layer was extracted with ethyl acetate and combined organic layer was washed with brine, dried over sodium sulphate, filtered, and concentrated. The residue was purified by silica gel chromatography, eluting with a gradient of hexanes providing 1-(3-chloro-5-methoxyphenyl) ethan-1-one as a yellow oily liquid (3.2 g, 80% yield). 1HNMR 400 MHz, DMSO-d6: δ 7.49 (t, J=4 Hz, 1H), 7.36 (d, J=4 Hz, 1H), 7.08 (t, J=4 Hz, 1H), 3.85 (s, 3H), 2.57 (s, 3H).

### Step-2: Synthesis of N—((R)-1-(3-chloro-5-methoxyphenyl)ethyl)-2-methylpropane-2-sulfinamide

To the solution of 1-(3-chloro-5-methoxyphenyl) ethan-1-one (2.0 g, 0.01 mol, 1.0 eq) and (R)-2-methylpropane-2-sulfinamide (2.6 g, 0.02 mol, 2.0 eq) in anhydrous tetrahydrofuran (20 ml) at 0° C., was added titanium tetraisopropoxide (5.5 g, 0.01 mol, 1.8 eq). The reaction was heated at 60° C. for 16 h. The reaction was cooled to −78° C. and sodium borohydride (1.2 g, 0.03 mol, 3.0 eq) was added and allowed to stir for 16 h at room temperature. The reaction was poured over crushed ice and extracted with ethyl acetate. The combined organic layer was washed with brine, dried over sodium sulphate, filtered and concentrated. The residue was purified by silica gel chromatography, eluting with a gradient of 0 to 70% ethyl acetate in hexanes providing N—((R)-1-(3-chloro-5-methoxyphenyl)ethyl)-2-methylpropane-2-sulfinamide as a white solid (1.1 g, 62% yield).

### Step-3: Synthesis of (R)-1-(3-chloro-5-methoxyphenyl) ethan-1-aminehydrochloride

To the solution of N—((R)-1-(3-chloro-5-methoxyphenyl)ethyl)-2-methylpropane-2-sulfinamide (0.5 g, 0.001 mol, 1.0 eq) in dichloromethane (20 ml) in a reaction vial was added 4M HCl in dioxane (5 ml) at 0° C. and stirred at room temperature for overnight. The reaction was concentrated and co-precipitated with MeOH: ether as co-solvent to provide (R)-1-(3-chloro-5-methoxyphenyl) ethan-1-aminehydrochloride as a white solid (0.4 g, 62% yield). 1HNMR 400 MHz, DMSO-d6: δ 8.59 (s, 3H), 7.17 (m, 2H), 7.02 (d, J=4 Hz, 1H), 4.38-4.35 (m, 1H), 3.80 (s, 3H), 1.49 (d, J=8 Hz, 1H).

### Step-4: Synthesis of (R)—N-(1-(3-chloro-5-methoxyphenyl)ethyl)-3-(trifluoromethyl)-[1,2,4]triazolo[4,3-b]pyridazin-6-amine

To the solution of 6-chloro-3-(trifluoromethyl)-[1,2,4]triazolo[4,3-b] pyridazine (0.26 g, 0.001 mol, 1.2 eq) in DMF (10 ml) in a reaction vial was added N, N-diisopropylethylamine (0.47 ml, 0.002 mol, 5.0 eq) followed by (R)-1-(3-chloro-5-methoxyphenyl) ethan-1-aminehydrochloride (0.2 g, 0.0009 mol, 1.0 eq). Reaction was sealed and heated at 80° C. for 16 h. The reaction was diluted with ethyl acetate and washed with brine, dried over sodium sulphate, filtered and concentrated. The residue was purified by silica gel chromatography, eluting with a gradient of 0 to 70% ethyl acetate in hexanes providing (R)—N-(1-(3-chloro-5-methoxyphenyl)ethyl)-3-(trifluoromethyl)-[1,2,4]triazolo[4,3-b]pyridazin-6-amine as a dark brown solid (0.12 g, 35% yield). LCMS: (m/z=372.21 [M+H]+); HPLC: 96.83%; Specific Optical Rotation: +318.9 (0.1% in MeOH)

1HNMR 400 MHz, DMSO-d6: δ 8.26 (d, J=7.2 Hz, 1H), 8.09 (d, J=10 Hz, 1H), 7.03-7.00 (m, 2H), 6.90 (t, J=2 Hz, 1H), 6.87 (t, J=2 Hz, 1H), 4.80-4.77 (m, 1H), 3.75 (s, 3H), 1.48 (d, J=6.8 Hz, 3H).

### Example 11: Synthesis of (R)—N-(1-(3,5-dichlorophenyl)ethyl)-3-(trifluoromethyl)-[1,2,4]triazolo[4,3-b]pyridazin-6-amine (Compound 8)

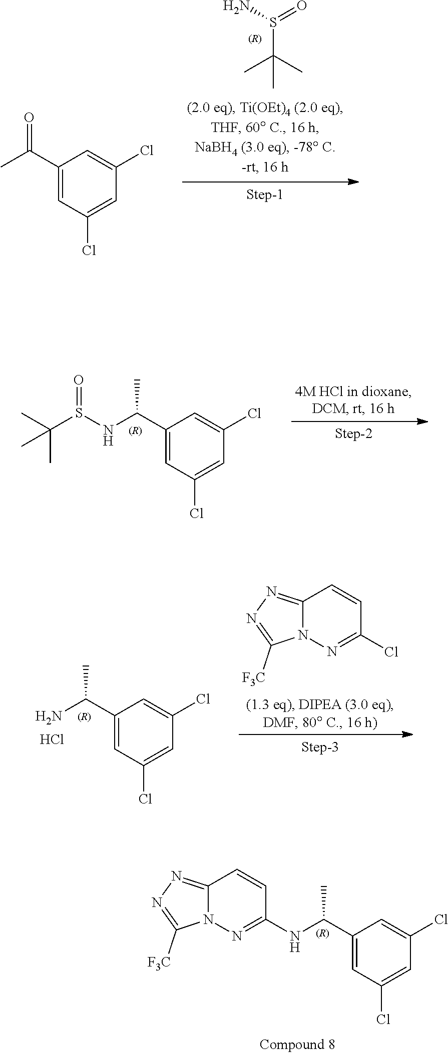

### Step-1: Synthesis of N—((R)-1-(3,5-dichlorophenyl)ethyl)-2-methylpropane-2-sulfinamide

To the solution 1-(3,5-dichlorophenyl) ethan-1-one (4.0 g, 0.0212 mol, 1.0 eq) and (R)-2-methylpropane-2-sulfinamide (5.0 g, 0.038 mol, 1.8 eq) in anhydrous tetrahydrofuran (100 ml) at 0° C., was added titanium tetraethoxide (25 ml, 0.0851 mol, 4.0 eq). The reaction was heated at 60° C. for 16 h. The reaction was cooled to −78° C. and sodium borohydride (2.43 g, 0.064 mol, 3.0 eq) was added and allowed to stir 16 h at room temperature. The reaction was poured over crushed ice and extracted with ethyl acetate. The combined organic layer was washed with brine, dried over sodium sulphate, filtered and concentrated. The residue was purified by silica gel chromatography, eluting with a gradient of 0 to 50% ethyl acetate in hexanes providing N—((R)-1-(4-fluoro-3-methoxyphenyl)ethyl)-2-methylpropane-2-sulfinamide as a white solid (3.5 g, 56% yield). 1H NMR (400 MHz, CDCl3): δ 7.28 (t, J=8 Hz, 1H), 7.23 (s, 1H), 7.20 (s, 1H), 4.51-4.45 (m, 1H), 3.44 (bs, 1H), 1.50 (d, J=8 Hz, 3H), 1.24 (s, 9H).

### Step-2: Synthesis of (R)-1-(3,5-dichlorophenyl) ethan-1-amine hydrochloride

To the solution of N—((R)-1-(3,5-dichlorophenyl)ethyl)-2-methylpropane-2-sulfinamide (3.5 g, 0.0120 mol, 1.0 eq) in dichloromethane (30 ml) in a reaction vial was added 4M HCl in dioxane (20 ml) at 0° C. and stirred at room temperature for 16 h. The reaction was concentrated and co-precipitated with MeOH: ether as co-solvent to provide (R)-1-(3,5-dichlorophenyl) ethan-1-amine hydrochloride as a white solid (0.2 g, 22% yield). 1HNMR 400 MHz, DMSO-d6: δ 8.75 (bs, 3H), 7.68-7.62 (m, 3H), 4.48-4.44 (m, 1H), 1.50 (d, J=8.0 Hz, 3H).

### Step-3: Synthesis of (R)—N-(1-(3,5-dichlorophenyl)ethyl)-3-(trifluoromethyl)-[1,2,4]triazolo[4,3-b]pyridazin-6-amine

To the solution of 6-chloro-3-(trifluoromethyl)-[1,2,4]triazolo[4,3-b] pyridazine (0.34 g, 2.222 mol, 1.0 eq) in DMF (10 ml) in a reaction vial was added N,N-diisopropylethylamine (0.78 ml, 6.66 mol, 3.0 eq) followed by (R)-1-(3,5-dichlorophenyl) ethan-1-amine hydrochloride (0.5 g, 2.222 mol, 3.0 eq). Reaction was sealed and heated at 80° C. for 16 h. The reaction was diluted with ethyl acetate and washed with brine, dried over sodium sulphate, filtered and concentrated. The residue was purified by silica gel chromatography, eluting with a gradient of 0 to 70% ethyl acetate in hexanes providing (R)—N-(1-(3,5-dichlorophenyl)ethyl)-3-(trifluoromethyl)-[1,2,4]triazolo[4,3-b] pyridazin-6-amine as a white color solid (0.1 g, 12% yield). LCMS: (m/z=377.57 [M+H]+); HPLC: 99.38%; Specific Optical Rotation: +310.12 (c 0.1% in MeOH)

1HNMR 400 MHz, DMSO-d6: δ 8.31 (d, J=6.4 Hz, 1H), 8.10 (d, J=10 Hz, 1H), 7.46-7.42 (m, 3H), 7.02 (d, J=10 Hz, 1H), 4.82-4.76 (m, 1H), 1.50 (d, J=6.8 Hz, 3H).

### Example 13: Synthesis of (R)-4-(1-((3-(trifluoromethyl)-[1,2,4]triazolo[4,3-b]pyridazin-6-yl)amino)ethyl)benzonitrile (Compound 9)

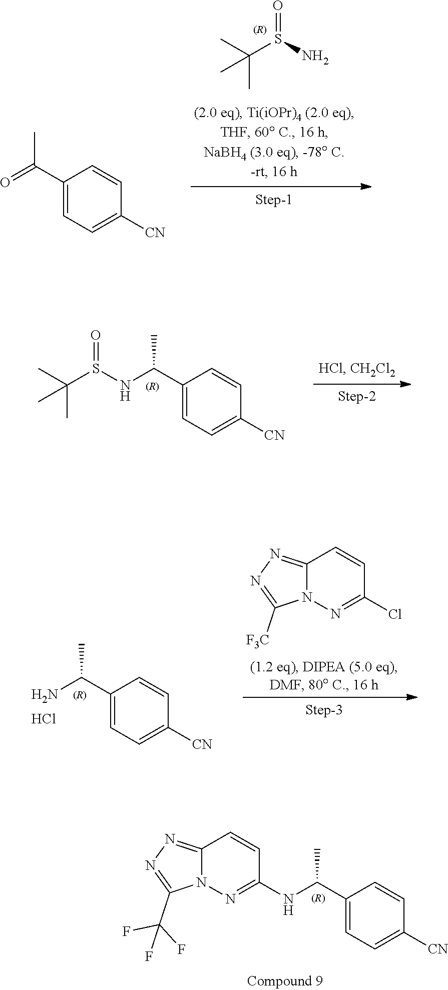

### Step-1: Synthesis of N—((R)-1-(4-cyanophenyl)ethyl)-2-methylpropane-2-sulfinamide

To the solution of 4-acetylbenzonitrile (2.0 g, 0.013 mol, 1.0 eq) and (R)-2-methylpropane-2-sulfinamide (3.30 g, 0.027 mol, 2.0 eq) in anhydrous tetrahydrofuran (40 mL) at 0° C., was added titanium tetraisopropoxide (5.50 g, 0.026 mol, 2.0 eq). The reaction was heated at 60° C. for 16 h. The reaction was cooled to −78° C. and sodium borohydride (1.5 g, 0.041 mol, 3.0 eq) was added and allowed to stir 16 h at room temp. The reaction was poured over crushed ice and extracted with ethyl acetate. The combined organic layer was washed with brine, dried over sodium sulphate, filtered and concentrated. The residue was purified by silica gel chromatography, eluting with a gradient of 0 to 50% ethyl acetate in hexanes providing N—((R)-1-(4-cyanophenyl)ethyl)-2-methylpropane-2-sulfinamide as a white solid (1.4 g; 40% yield). 1H NMR (400 MHz, DMSO-d6): δ 7.65 (d, J=8.0 Hz, 2H), 7.47 (d, J=8.0 Hz, 2H), 4.60-4.57 (m, 1H), 1.14-4.09 (m, 1H), 1.52 (d, J=8 Hz, 3H), 1.24 (s, 9H).

### Step-2: Synthesis of (R)-4-(1-aminoethyl)benzonitrile hydrochloride

To the solution of N—((R)-1-(4-cyanophenyl)ethyl)-2-methylpropane-2-sulfinamide (0.25 g, 0.001 mol, 1.0 eq) in dichloromethane (20 ml) in a reaction flask was added 4M HCl in dioxane (5 ml) at 0° C. and stirred at room temperature for overnight. The reaction was concentrated and co-precipitated with MeOH: Di-ethyl ether as co-solvent to provide (R)-4-(1-aminoethyl)benzonitrile hydrochloride as a white solid (0.16 g; 87% yield). 1H NMR (400 MHz, DMSO-d6): δ 8.63 (s, 3H), 7.93 (d, J=8.0 Hz, 2H), 7.72 (d, J=8.0 Hz, 1H), 4.54-4.48 (m, 1H), 1.49 (d, J=8.0 Hz, 3H).

### Step-3: Synthesis of (R)-4-(1-((3-(trifluoromethyl)-[1,2,4]triazolo[4,3-b]pyridazin-6-yl)amino)ethyl)benzonitrile

To the solution of 6-chloro-3-(trifluoromethyl)-[1,2,4]triazolo[4,3-b] pyridazine (0.23 g, 0.001 mol, 1.2 eq) in DMF (10 ml) in a reaction vial was added N,N-diisopropylethyl amine (1.0 ml, 0.004 mol, 5.0 eq) followed by (R)-1-(3-chloro-5-methoxyphenyl) ethan-1-aminehydrochloride (5, 0.16 g, 0.008 mol, 1.0 eq). Reaction was sealed and heated at 80° C. for 16 h. The reaction was diluted with ethyl acetate and washed with brine, dried over sodium sulphate, filtered, and concentrated. The residue was purified by silica gel chromatography, eluting with a gradient of 0 to 70% ethyl acetate in hexanes providing (R)-4-(1-((3-(trifluoromethyl)-[1,2,4]triazolo[4,3-b]pyridazin-6-yl)amino)ethyl)benzonitrile as cream color solid (0.1 g; 28% yield). HPLC: 99.87%; LCMS: (m/z=333.19 [M+H]+); Specific Optical Rotation: +435.32 (c 0.1% in MeOH).

1H NMR (400 MHz, DMSO-d6): δ 8.40 (d, J=6.4 Hz, 1H), 8.10 (d, 1H, J=10.0 Hz, 1H), 7.78-7.77 (m, 2H), 7.57-7.55 (m, 2H), 7.05 (d, J=10.0 Hz, 1H), 4.91-4.84 (m, 1.0 Hz), 1.50 (d, J=6.8 Hz, 3H).

### Example 14: Synthesis of (R)—N-(1-(4-fluorophenyl)ethyl)-3-methyl-[1,2,4]triazolo[4,3-b]pyridazin-6-amine (Compound 10)

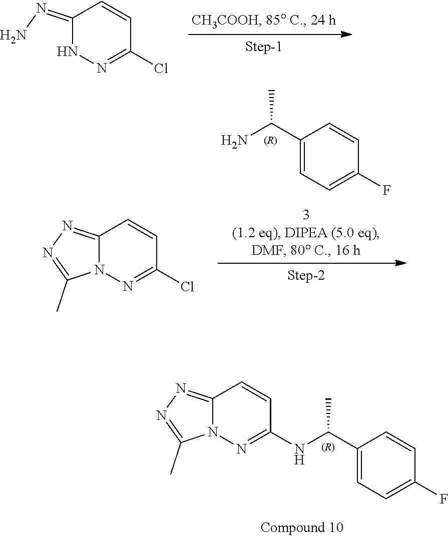

### Step-1: Synthesis of 6-chloro-3-methyl-[1,2,4]triazolo[4,3-b]pyridazine

3-chloro-6-hydrazinopyridazine (5.0 g, 0.034 mol, 1.0 eq) was taken in a sealed tube and acetic acid (100 ml) was added. The reaction mixture was heated at 85° C. for 24 h. The reaction mixture was evaporated to dryness. The crude mass was dissolved in ethyl acetate and washed with aqueous solution of sodium bicarbonate three times. The organic layer was separated, and the aqueous layer was extracted with ethyl acetate twice. The combined organic layers were washed with brine, dried over sodium sulphate, filtered and concentrated under reduced pressure to get 6-chloro-3-methyl-[1,2,4]triazolo[4,3-b] pyridazine as an off brown solid (5.0 g, 86% yield). 1HNMR 400 MHz, DMSO-d6: δ 8.41 (d, J=8.0 Hz, 1H), 7.45 (d, J=8.0 Hz, 1H), 2.67 (s, 3H)

### Step-2: Synthesis of (R)—N-(1-(4-fluorophenyl)ethyl)-3-methyl-[1,2,4]triazolo[4,3-b]pyridazin-6-amine

To the solution of 6-chloro-3-methyl-[1,2,4]triazolo[4,3-b]pyridazine (0.5 g, 0.0034 mol, 1.0 eq) in DMF (10 ml) in a reaction vial was added N,N-diisopropylethylamine (2.1 ml, 0.02 mol, 5.0 eq) followed by (R)-1-(4-fluorophenyl) ethan-1-amine (0.627 g, 0.004 mol, 1.2 eq). Reaction was sealed and heated at 80° C. for 16 h. The reaction was diluted with ethyl acetate and washed with brine, dried over sodium sulphate, filtered and concentrated. The residue was purified by silica gel chromatography, eluting with a gradient of 0 to 70% ethyl acetate in hexanes providing (R)—N-(1-(4-fluorophenyl)ethyl)-3-methyl-[1,2,4]triazolo[4,3-b]pyridazin-6-amine as a white solid (0.09 g, 9.1% yield). LCMS: (m/z=270.12 [M−H]+); HPLC: 99.65%; Specific Optical Rotation: +357.53 (c 0.1% in MeOH)

1NMR: HNMR 400 MHz, DMSO-d6: δ 7.84 (d, J=5.6 Hz, 1H), 7.80 (t, J=6.8 Hz, 1H), 7.47-7.43 (m, 2H), 7.16-7.11 (m, 2H), 6.78 (d, J=9.6 Hz, 1H), 4.95-4.87 (m, 1H), 2.42 (s, 3H), 1.47 (d, J=5.8 Hz, 3H).

### Example 15: Synthesis of (R)-3-cyclopropyl-N-(1-(4-fluorophenyl)ethyl)-[1,2,4]triazolo[4,3-b]pyridazin-6-amine (Compound 11)

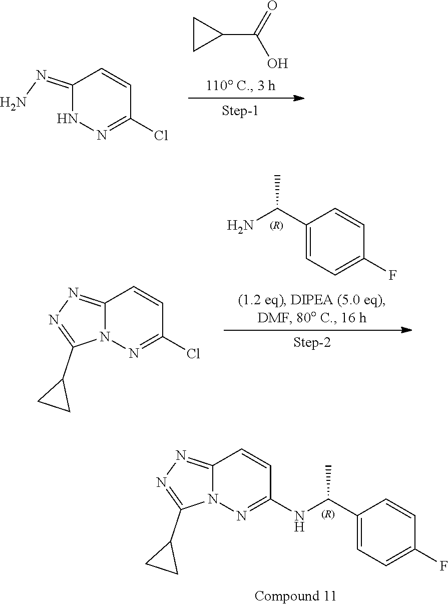

### Step-1: Synthesis of 6-chloro-3-cyclopropyl-[1,2,4]triazolo[4,3-b]pyridazine

3-chloro-6-hydrazinopyridazine (2.0 g, 0.012 mol, 1.0 eq) was taken in a sealed tube and cyclopropyl acetic acid (10 ml) was added. The reaction mixture was heated at 110° C. for 3 h. The reaction mixture was evaporated to dryness. The crude mass was dissolved in ethyl acetate and washed with aqueous solution of sodium bicarbonate three times. The organic layer was separated, and the aqueous layer was extracted with ethyl acetate twice. The combined organic layers were washed with brine, dried over sodium sulphate, filtered and concentrated under reduced pressure to get 6-chloro-3-cyclopropyl-[1,2,4]triazolo[4,3-b]pyridazine as a brown solid (0.6 g, 26% yield). 1H NMR (400 MHz, CDCl3): δ 8.01 (d, J=4.0 Hz, 1H), 7.05 (d, J=8.0 Hz, 1H), 2.46-2.44 (m, 1H), 1.38-1.35 (m, 2H), 1.25-1.21 (m, 2H).

### Step-2: Synthesis of (R)-3-cyclopropyl-N-(1-(4-fluorophenyl)ethyl)-[1,2,4]triazolo[4,3-b]pyridazin-6-amine

To the solution of 6-chloro-3-cyclopropyl-[1,2,4]triazolo[4,3-b]pyridazine (0.6 g, 0.003 mol, 1.0 eq) in DMF (20 ml) in a reaction vial was added N,N-diisopropylethylamine (2.6 ml, 0.015 mol, 5.0 eq) followed by (R)-1-(4-fluorophenyl) ethan-1-amine (0.6 g, 0.0036 mol, 1.2 eq). Reaction was sealed and heated at 80° C. for 16 h. The reaction was diluted with ethyl acetate and washed with brine, dried over sodium sulphate, filtered and concentrated. The residue was purified by silica gel chromatography, eluting with a gradient of 0 to 70% ethyl acetate in hexanes providing (R)-3-cyclopropyl-N-(1-(4-fluorophenyl)ethyl)-[1,2,4]triazolo[4,3-b]pyridazin-6-amine as a white solid (0.04 g, 4.3%). LCMS: (m/z=296.09 [M−H]+); HPLC: 94.4%; Specific Optical Rotation: +331.21 (c 0.1% in MeOH)

1HNMR 400 MHz, DMSO-d6: δ 7.82 (d, J=6.8 Hz, 2H), 7.47-7.42 (m, 2H), 7.17-7.14 (m, 2H), 6.77 (d, J=10 Hz, 2H), 4.92-4.85 (m, 1H), 2.23-2.16 (m, 1H), 1.48 (d, J=7.2 Hz, 3H), 1.08-1.03 (m, 2H), 1.02-0.96 (m, 2H).

### Example 16: Synthesis of ((R)-3-(difluoromethyl)-N-(1-(4-fluorophenyl)ethyl)-[1,2,4]triazolo[4,3-b]pyridazin-6-amine (Compound 12)

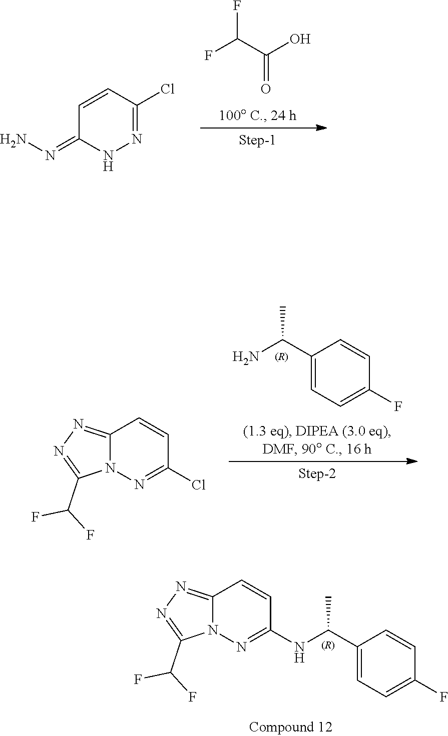

### Step-1: Synthesis of 6-chloro-3-(difluoromethyl)-[1,2,4]triazolo[4,3-b]pyridazine

3-chloro-6-hydrazinopyridazine (1.0 g, 0.006 mol, 1.0 eq) was taken in a sealed tube and 2,2-difluoroacetic acid (10 ml) was added. The reaction mixture was heated at 100° C. for 24 h. The reaction mixture was evaporated to dryness. The crude mass was dissolved in ethyl acetate and washed with aqueous solution of sodium bicarbonate three times. The organic layer was separated, and the aqueous layer was extracted with ethyl acetate twice. The combined organic layers were washed with brine, dried over sodium sulphate, filtered and concentrated under reduced pressure to get 6-chloro-3-(difluoromethyl)-[1,2,4]triazolo[4,3-b]pyridazine as an off brown solid (1.0 g, 70% yield). LCMS: (m/z=205.2 [M+H]+)

### Step-2: Synthesis of ((R)-3-(difluoromethyl)-N-(1-(4-fluorophenyl)ethyl)-[1,2,4]triazolo[4,3-b]pyridazin-6-amine

To the solution of 6-chloro-3-(difluoromethyl)-[1,2,4]triazolo[4,3-b]pyridazine (0.4 g, 0.0019 mol, 1.0 eq) in DMF (10 ml) in a reaction vial was added N, N-diisopropylethylamine (1.1 ml, 0.0038 mol, 3.0 eq) followed by (R)-1-(4-fluorophenyl) ethan-1-amine (1.6 g, 0.015 mol, 1.3 eq). Reaction was sealed and heated at 90° C. for 16 h. The reaction was diluted with ethyl acetate and washed with brine, dried over sodium sulphate, filtered and concentrated. The residue was purified by silica gel chromatography, eluting with a gradient of 0 to 5% MeOH in dichloromethane providing ((R)-3-(difluoromethyl)-N-(1-(4-fluorophenyl)ethyl)-[1,2,4]triazolo[4,3-b]pyridazin-6-amine as a cream color solid (0.1 g, 16% yield). LCMS: (m/z=307.79 [M+H]+); HPLC: 99.7%; Specific Optical Rotation: +333.50 (c 0.1% in MeOH)

1HNMR 400 MHz, DMSO-d6: δ 8.10 (d, J=7.2 Hz, 1H), 8.01 (d, J=10.0 Hz, 1H), 7.48-7.44 (m, 2H), 7.40 (t, J=52 Hz, 1H), 7.16-7.11 (m, 2H), 6.94 (d, J=10.0 Hz, 1H), 4.93-4.86 (m, 1H), 1.49 (d, J=6.8 Hz, 3H).

### Biological Studies

### Indigo Biosciences Discovery Assay

The aim of this study was to evaluate compounds 1-12 and the comparative compounds 1-2 for inhibition activities against human AR. Reporter Cells used in these assays expressed a native receptor (AR). The reporter gene, firefly luciferase, is functionally linked to an upstream receptor-specific genetic response element (GRE). Reporter cells were treated with eight concentrations, starting at 50 μM and following with 3.17-fold dilutions. Single treatment concentration was performed (n=1). Assay performance was validated using the reference antagonist hydroxy flutamide.
Step 1: A suspension of reporter cells was prepared in Cell Recovery Medium (CRM). Reporter cells were first supplemented with 2×-EC80 concentration of the reference agonist, 5α-dihydro-11-keto testosterone, then 100 μl of the Reporter Cell suspension was dispensed into wells of a white 96-well assay plate.
Step 2: Test compound master stocks were diluted in DMSO to generate solutions at ‘500×-concentration’ relative to the final treatment concentration and diluted directly into INDIGO's Compound Screening Medium (CSM; containing charcoal-stripped FBS) to generate ‘2×-concentration’ treatment media. This was diluted into the wells to the final concentration. Assay plates were incubated at 37° C., 5% CO₂ and ~70% humidity for 24 hr.
Step 3: Following the incubation period, wells were rinsed once with Live Cell Multiplex (LCM) Buffer, then LCM substrate was added. Following incubation at room temperature for 15 min, LCM substrate was discarded and 100 μL/well of luciferase detection reagent was added. Subsequently, fluorescence was measured to determine the relative number of live cells per assay well. RLU values were quantified after a 10 min incubation period at room temperature to determine nuclear receptor activities.

### Eurofins AR Radioligand Binding Assay

Human androgen receptors obtained from human LNCaP cells were used in modified HEPES buffer pH 7.4. A 70 μg‡ aliquot was incubated with 0.5 nM [3H] Methyltrienolone for 20 h at 4° C. Non-specific binding was estimated in the presence of 1 μM testosterone. Receptors were filtered and washed, the filters were then counted to determine [3H] Methyltrienolone specifically bound. Compounds were screened at 10 μM.

### Mouse/Human Liver Microsome Stability

### Procedure Summary

| Test System | Liver Microsomes | |
|---|---|---|
| Test compound concentration | 1 | μM |
| Time Points | 0, 5, 10, 30 and 60 minutes | |
| No of Replicates | Two | |
| Final Protein Concentration | 1 | mg/mL |
| Final NADPH Concentration | 1 | mM |
| Potassium Phosphate Buffer pH 7.4 | 100 | mM |

| | |
|---|---|
| Final DMSO Concentration | <0.1% |
| Deliverables | % Remaining of test compound, |
| | Half life, CLint |
| Bioanalysis | LC-MS/MS |

### Assay Procedure

Preincubation mixture 2.5 μL Test Cpd. +75 μL Liver microsomes (human or mouse source) @ 3.33 mg/mL+85 L of 100 mM potassium phosphate buffer (preincubate for 10 min @ 37° C.). Incubation mixture 62 μL of cofactor (2.85 mM)+Remaining incubation mixture (Incubated for 60 min @ 37° C.). Sample preparation 25 μL incubation mixture+200 μL of acetonitrile containing internal standard+Vortex 5 min @ 1200 rpm+Centrifuge 10 min @ 4000 rpm. Supernatant diluted 2-fold with water and injected on LC-MS/MS.

Table 1 shows the AR inhibition indigo cell based average IC50 (nM) values for Compound 1 and Comparative Compounds 1-2.

TABLE 1
AR inhibition indigo cell based average IC50 (nM) values
for Compound 1 and Comparative Compounds 1 and 2

| | AR Inhibition Indigo |
|---|---|
| | Cell Based: Avg IC50 |
| Compound | (nM)* |
| Comparative Compound 1 | ++ |
| Comparative Compound 2 | +++ |
| Compound 1 | + |

*0-50 nm: “+”; 51 nm-500 nm: “++”, >501 nm: “+++”

Table 2 shows the AR inhibition indigo cell based average IC50 (nM) values for Compounds 1-12.

TABLE 2
AR inhibition indigo cell based average
IC50 (nM) values for Compounds 1-12

| | AR Inhibition Indigo |
|---|---|
| | Cell Based: Avg IC50 |
| Compound | (nM)* |
| Compound 1 | + |
| Compound 2 | + |
| Compound 3 | + |
| Compound 4 | ++ |
| Compound 5 | ++ |
| Compound 6 | ++ |
| Compound 7 | ++ |
| Compound 8 | ++ |
| Compound 9 | +++ |
| Compound 10 | ++ |
| Compound 11 | +++ |
| Compound 12 | ++ |

*0-50 nm: “+”; 51 nm-500 nm: “++”, >501 nm: “+++”

Table 3 shows the AR antagonist biochemical average IC50 (nM) values for Compound 1 and Comparative Compounds 1-2.

TABLE 3
AR antagonist biochemical average IC50 (nM) values
for Compound 1 and Comparative Compounds 1 and 2

| | Biochemical AR LBD |
|---|---|
| | Antagonism: Avg IC50 |
| Compound | (nM)* |
| Comparative Compound 1 | +++ |
| Comparative Compound 2 | +++ |
| Compound 1 | +++ |

*0-50 nm: “+”; 51 nm-500 nm: “++”, >501 nm: “+++”

Table 4 shows the AR antagonist biochemical average IC50 (nM) values for Compounds 1-12.

TABLE 4
AR antagonist biochemical average
IC50 (nM) values for Compounds 1-12

| | Biochemical AR LBD |
|---|---|
| | Antagonism: |
| Compound | Average IC50 (nM) * |
| Compound 1 | +++ |
| Compound 2 | +++ |
| Compound 3 | +++ |
| Compound 4 | ND |
| Compound 5 | ++ |
| Compound 6 | +++ |
| Compound 7 | ++ |
| Compound 8 | +++ |
| Compound 9 | ND |
| Compound 10 | +++ |
| Compound 11 | ND |
| Compound 12 | +++ |

*0-50 nm: “+”; 51 nm-500 nm: “++”, >501 nm: “+++”

Table 5 shows the in vitro microsome stability data for Compound 1 and Comparative Compounds 1 and 2.

TABLE 5
In vitro microsome stability data for compound
1 and comparative compounds 1-2

| | Human LM: Rem @ | Mouse LM: Rem @ |
|---|---|---|
| Compound | 60 min (Percent)* | 60 min (Percent)* |
| Compound 1 | +++ | ++ |
| Comparative | +++ | +++ |
| Compound 1 | | |
| Comparative | NA | NA |
| Compound 2 | | |

*0-30: “+”; 30.1-60: “++”, >60.1: “+++”

Table 6 shows the in vitro microsome stability data for Compounds 1-12

TABLE 6
In vitro microsome stability data for compounds 1-12

| | Human LM: Rem @ | Mouse LM: Rem @ |
|---|---|---|
| Compound | 60 min (Percent)* | 60 min (Percent)* |
| Compound 1 | +++ | ++ |
| Compound 2 | +++ | +++ |
| Compound 3 | +++ | +++ |
| Compound 4 | +++ | +++ |
| Compound 5 | +++ | + |
| Compound 6 | +++ | + |
| Compound 7 | +++ | + |
| Compound 8 | +++ | +++ |
| Compound 9 | ND | ND |
| Compound 10 | +++ | +++ |
| Compound 11 | ND | ND |
| Compound 12 | +++ | +++ |

As shown in Table 1, inventors of the present invention have unexpectedly found that an R isomer (Compound 1) was ~150 times more potent than the S isomer (Comparative Compound 2) in the cell-based activity assay. Further, the R isomer (Compound 1) was ~2 times more potent than the racemic mixture (Comparative Compound 1) in the cell-based activity assay. This indicates an unpredictable biological preference for R-isomer stereochemistry with respect to AR modulation for the compounds described in the present application. This stereo-selectivity is unexpected and allows for a more active pharmacological treatment of AR-related cancers. Moreover, the ability to use only the most-active R form may be beneficial in comparison to the racemic mixture. Dosing with the active isomer minimizes the metabolic burden of the xenobiotic (drug) and the potential for toxicity that could be caused by dosing with the unnecessary isomer, which has been observed for other pharmaceutics.

Furthermore, as noted earlier, most clinically relevant AR inhibitors target the ligand binding domain (LBD) of the androgen receptor, and these inhibitors can lose efficacy due to point mutations in the LBD. Given that the AR protein contains multiple sub-domains, targeting other domains is an attractive approach for new inhibitors. As shown in Tables 1 and 2, the compounds as described herein are effective at inhibiting androgen pathway activity as indicated by the reduction of an AR-driven luciferase reporter system in live cells. This demonstrates activity against the intended pathway, but does not identify what portion of the AR they interact with. The data in Tables 3 and 4 highlight that these nM inhibitors in the cell base system (with impediments of cell membranes and binding to media proteins) are much less potent when assessed for their ability to function as an LBD-antagonist in a biochemical assay (with just AR-LBD, the compounds, and DHT). Therefore, the compounds as described herein may be interacting with other subdomains in the AR protein, namely the N-terminal domain.

Further, Tables 5 and 6 demonstrate that the compounds as described herein have suitable metabolic stability in vitro for pre-clinical studies (Mouse LM) and equal or better stability in human liver microsomes suggesting good potential for therapeutic use.

While only certain features of several embodiments have been illustrated and described herein, many modifications and changes will occur to those skilled in the art. It is, therefore, to be understood that the appended claims are intended to cover all such modifications and changes as fall within the scope of the invention and the appended claims.

1. A compound having a formula (I) or a pharmaceutically acceptable salt thereof: 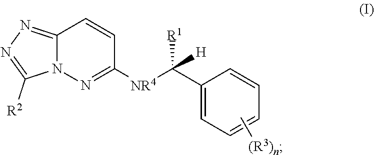
wherein
R¹ and R² are independently a C₁-C₆ alkyl group optionally substituted with one or more fluorine or a C₃-C₆ cycloalkyl group optionally substituted with one or more fluorine;
R³ is independently at each occurrence halogen, —CN, —OR⁵, —C(═O)R⁵, —NHC(═O)R⁵, —C(═O)NR⁵R⁶, —NR⁵R⁶, —P(—O)—R⁵R⁶, a C₁-C₆ alkyl group optionally substituted with one or more fluorine, or a C₃-C₆ cycloalkyl group optionally substituted with one or more fluorine;
R⁴ is hydrogen or a C₁-C₆ alkyl group;
R⁵ and R⁶ are independently at each occurrence hydrogen, a C₁-C₆ alkyl group, or
R⁵ and R⁶ together with the nitrogen to which each is attached form a C₃-C₆ heterocycloalkyl group optionally substituted with one or more fluorine; and
“n” is an integer from 1 to 5.

2. The compound of claim 1, wherein R¹ is a C₁-C₆ alkyl group.

3. The compound of claim 1, wherein R² is a C₁-C₆ alkyl group substituted with one or more fluorine.

4. The compound of claim 1, wherein R¹ is a C₁-C₆ alkyl group, and R³ is independently at each occurrence halogen, —CN, —OR⁴, or a C₁-C₆ alkyl group optionally substituted with one or more fluorine.

5. The compound of claim 1, wherein R² is a C₁-C₆ alkyl group substituted with one or more fluorine, and R³ is independently at each occurrence halogen, —CN, —OR⁴, or a C₁-C₆ alkyl group optionally substituted with one or more fluorine.

6. The compound of claim 1, wherein R¹ is a C₁-C₆ alkyl group, R² is a C₁-C₆ alkyl group substituted with one or more fluorine, and R³ is independently at each occurrence halogen, —CN, —OR⁴, or a C₁-C₆ alkyl group optionally substituted with one or more fluorine.

7. The compound of claim 1, wherein R¹ is a C₁-C₆ alkyl group, R² is a C₁-C₆ alkyl group substituted with one or more fluorine, and R³ is independently at each occurrence halogen.

8. The compound of claim 1 having a formula (II) or a pharmaceutically acceptable salt thereof: 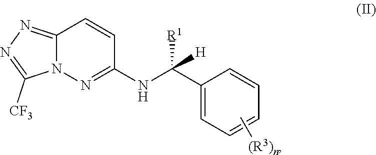
wherein R¹ is a C₁-C₆ alkyl group, R³ is independently at each occurrence halogen, —CN, —OR⁵, or a C₁-C₆ alkyl group optionally substituted with one or more fluorine, and “n” is an integer from 1 to 5.

9. The compound of claim 1 having a formula (III) or a pharmaceutically acceptable salt thereof: 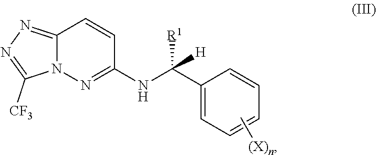
wherein R¹ is a C₁-C₆ alkyl group, X is halogen and “n” is an integer from 1 to 5.

10. The compound of claim 1 having a formula (IV) or a pharmaceutically acceptable salt thereof: 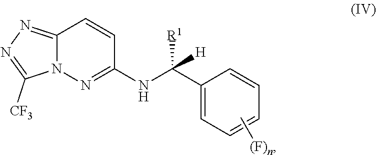
wherein R¹ is a C₁-C₆ alkyl group and “n” is an integer from 1 to 5.

11. The compound of claim 1 having a formula (V) or a pharmaceutically acceptable salt thereof: 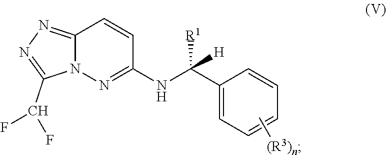
wherein R¹ is a C₁-C₆ alkyl group, R³ is independently at each occurrence halogen, —CN, —OR⁵, or a C₁-C₆ alkyl group optionally substituted with one or more fluorine, and “n” is an integer from 1 to 5.

12. The compound of claim 1 having a formula (VI) or a pharmaceutically acceptable salt thereof: 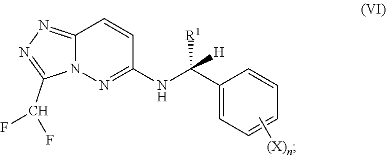
wherein R¹ is a C₁-C₆ alkyl group, X is halogen, and “n” is an integer from 1 to 5.

13. The compound of claim 1 having a formula (VII) or a pharmaceutically acceptable salt thereof: 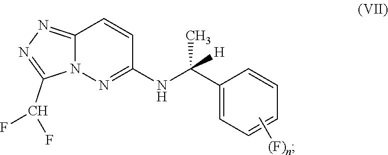
wherein R¹ is a C₁-C₆ alkyl group and “n” is an integer from 1 to 5.

14. The compound of claim 1 selected from the group consisting of: 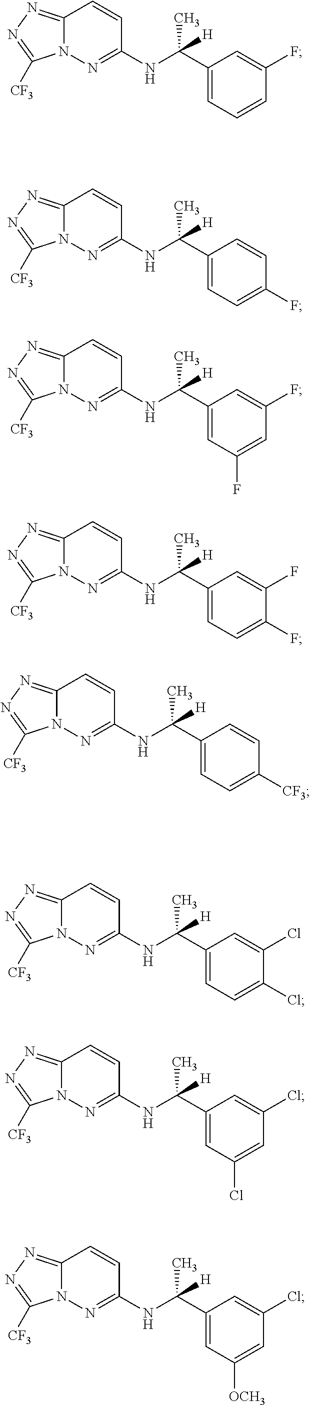 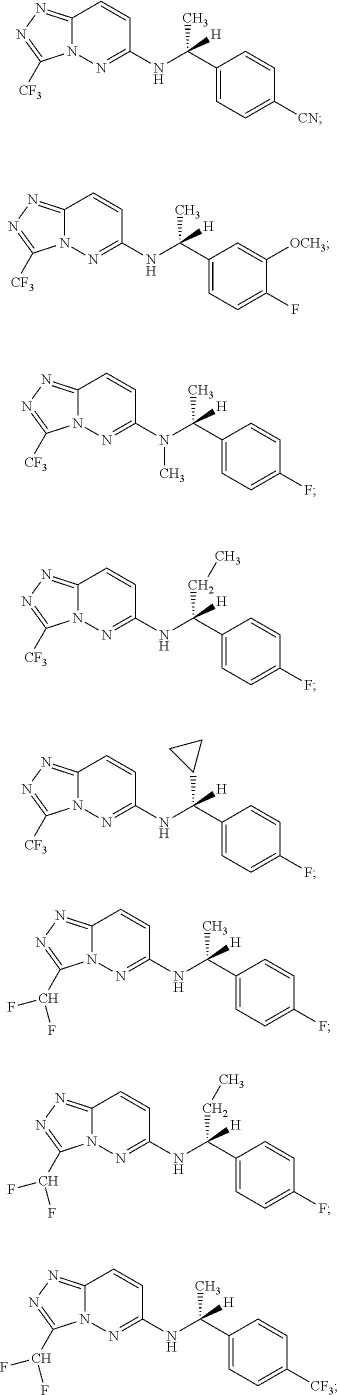 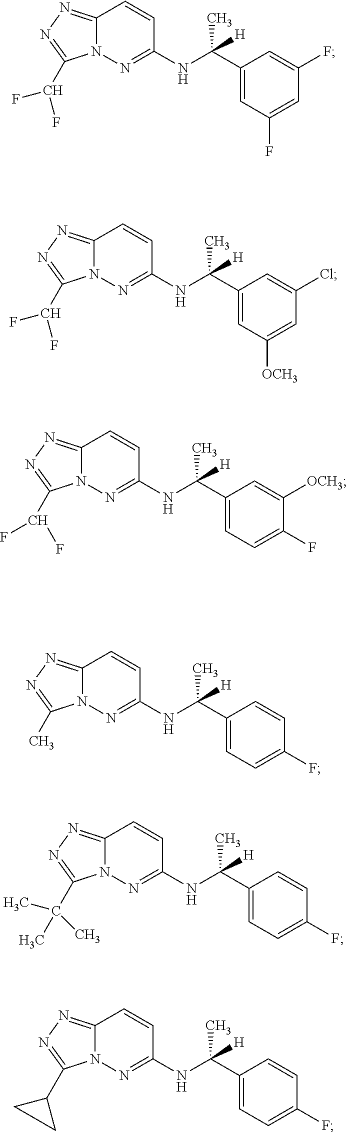 [SMILES: C[C@@H](Nc1ccc2nnc(C(F)F)n2n1)c1cc(F)cc(F)c1 ; COc1cc(Cl)cc([C@@H](C)Nc2ccc3nnc(C(F)F)n3n2)c1 ; COc1cc([C@@H](C)Nc2ccc3nnc(C(F)F)n3n2)ccc1F ; Cc1nnc2ccc(N[C@H](C)c3ccc(F)cc3)nn12 ; C[C@@H](Nc1ccc2nnc(C(C)(C)C)n2n1)c1ccc(F)cc1 ; C[C@@H](Nc1ccc2nnc(C3CC3)n2n1)c1ccc(F)cc1]
and any pharmaceutically acceptable salts thereof.

15. A pharmaceutical composition comprising:
the compound of claim 1 or a pharmaceutically acceptable salt, solvate, hydrate, polymorph, or co-crystal thereof, and
a pharmaceutically carrier, diluent, or excipient.

16. The pharmaceutical composition of claim 15, comprising a compound of formula (II) or (V) or a pharmaceutically acceptable salt, solvate, hydrate, polymorph, or co-crystal thereof: 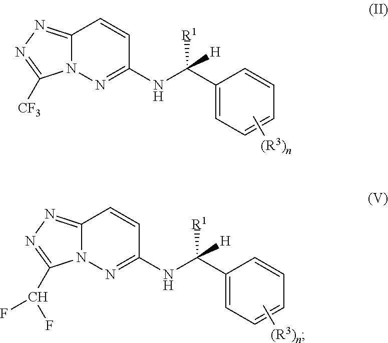
wherein R¹ is a C₁-C₆ alkyl group, R³ is independently at each occurrence halogen, —CN, —OR⁵, or a C₁-C₆ alkyl group optionally substituted with one or more fluorine, and “n” is an integer from 1 to 5; and
a pharmaceutically carrier, diluent, or excipient.

17. A method for modulation of androgen receptor activity for treatment of prostate cancer, breast cancer, ovarian cancer, or melanoma, the method comprising administering to a patient a therapeutically effective amount of a pharmaceutical composition comprising a compound of claim 1.

18. The method of claim 17, wherein the method comprises modulation of androgen receptor activity for treatment of metastatic castration-resistant prostate cancer.

19. The method of claim 18 wherein the method comprises modulation of N-terminal domain of an androgen receptor for treatment of metastatic castration-resistant prostate cancer.

20. The method of claim 17, wherein the method comprises administering to a patient a therapeutically effective amount of a pharmaceutical composition comprising a compound of formula (II) or (V): 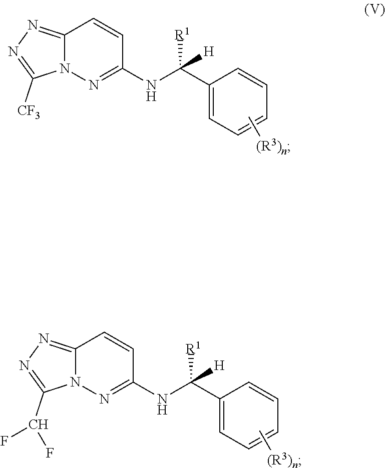
wherein R¹ is a C₁-C₆ alkyl group, R³ is independently at each occurrence halogen, —CN, —OR⁵, or a C₁-C₆ alkyl group optionally substituted with one or more fluorine, and “n” is an integer from 1 to 5.
